(19) United States
(12) Patent Application Publication
(10) Pub. No.: US 2026/0151444 A1
(43) Pub. Date: Jun. 4, 2026

(54) ADAPTER FOR UNIVERSAL TUMOR TARGETING

(71) Applicant: VIROGIN BIOTECH CANADA LTD, Richmond (CA)

(72) Inventors: Xiaohu Liu, Vancouver (CA); Yanal M. Murad, Vancouver (CA); I-Fang Lee, Vancouver (CA); William Wei-Guo Jia, Vancouver (CA)

(21) Appl. No.: 19/110,071

(22) PCT Filed: Sep. 8, 2023
(86) PCT No.: PCT/US2023/073811

§ 371 Date: Mar. 8, 2025

Related U.S. Application Data
(60) Provisional application No. 63/375,032, filed on Sep. 8, 2022.

(51) Int. Cl. A61K 35/763; A61K 40/11; A61K 40/31; A61K 40/42; A61K 40/46; A61P 35/00

(52) U.S. Cl. CPC A61K 35/763; A61K 40/11; A61K 40/31; A61K 40/4205; A61K 40/4211; A61K 40/46; A61P 35/00; C12N 2710/16622; C12N 2710/16632

(57) ABSTRACT

Compositions and methods are provided that enhance CAR T-cell therapy by providing an oncolytic virus engineered to direct expression and secretion of a multivalent adapter protein. The multivalent adapter protein targets both a broadly tumor-associated cell surface antigen and an antigen binding domain of a CAR T-cell.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to oncolytic viruses that direct expression of a soluble multivalent adapter/engager protein that enhances cell-based cancer therapies

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of VIRO_417P3_sequencelisting.text.xml; Size: 9.80 bytes; and Date of Creation: Jul. 11, 2022. The Sequence Listing filed via EFS-Web is part of the specification and is incorporated in its entirety by reference herein.

### BACKGROUND

T cells genetically engineered with chimeric antigen receptors (“CAR T-cells”) to target a specific protein on the surface of tumor cells are a potent tool for cancer immunotherapy. The chimeric antigen receptors (“CARs”) contain an extracellular domain from the single-chain variable fragment (“scFv”) region of an antibody specific to the tumor associated antigen being targeted, a transmembrane domain, and an intracellular domain from the signal activating regions for T cells. In contrast to T cells that are isolated and genetically modified in vitro to express TCRs that target specific tumor antigens (TCR-T), CAR T-cells recognize and kill tumor cells independently of the major histocompatibility complex (“MHC”), bypassing the numerous strategies employed by tumor cells to avoid MHC-restricted T-cell recognition (Li R, Ma C, Cai H, Chen W. The CAR T-Cell Mechanoimmunology at a Glance. Adv Sci (Weinh). 2020 Nov. 3; 7(24):2002628. doi: 10.1002/advs.202002628. PMID: 33344135; PMCID: PMC7740088). CAR T-cell therapies have demonstrated clinical success in treating hematological malignancies, resulting in FDA approval of therapies that utilize CARs targeting CD19 and BCMA, which are proteins typically expressed in B-cell leukemia/lymphoma and multiple myeloma, respectively. Multiple clinical trials are underway to evaluate CAR T-cells targeting a variety of novel antigens abundant in lymphomas and leukemias, including CD20, CD22, CD30, CD33, CD123, and FLT3 (Brudno J N, Kochenderfer J N. Chimeric antigen receptor T-cell therapies for lymphoma. Nat Rev Clin Oncol. 2018 January; 15(1):31-46. doi: 10.1038/nrclinonc.2017.128. Epub 2017 Aug. 31. PMID: 28857075) (Schultz L, Mackall C. Driving CAR T cell translation forward. Sci Transl Med. 2019 Feb. 27; 11(481):eaaw2127. doi: 10.1126/scitranslmed.aaw2127. PMID: 30814337) (Arabi F, Torabi-Rahvar M, Shariati A, Ahmadbeigi N, Naderi M. Antigenic targets of CAR T Cell Therapy. A retrospective view on clinical trials. Exp Cell Res. 2018 Aug. 1; 369(1):1-10. doi: 10.1016/j.yexcr.2018.05.009. Epub 2018 May 31. PMID: 29758187).

Despite these successes, adoptive cell therapies such as CAR-T cell therapy have not been particularly effective in treating solid tumors. This is largely due to the presence of barriers such as the tumor stroma and the immunosuppressive tumor microenvironment, which can limit CAR T-cell activation (Kakarla S, Gottschalk S. CAR T cells for solid tumors: armed and ready to go? Cancer J. 2014 March-April; 20(2):151-5. doi: 10.1097/PPO.0000000000000032. PMID: 24667962; PMCID: PMC4050065). Another fundamental issue is the lack of promising target antigens on the tumor surface that are not found on essential healthy tissues. Off-target effects are well documented in anti-CD19 CAR T-cell therapy because normal B-cells also express CD19, but the resulting B-cell aplasia can be managed with intermittent immunoglobulin replacement (Makita S, Yoshimura K, Tobinai K. Clinical development of anti-CD19 chimeric antigen receptor T-cell therapy for B-cell non-Hodgkin lymphoma. Cancer Sci. 2017 June; 108(6):1109-1118. doi: 10.1111/cas.13239. Epub 2017 May 25. PMID: 28301076; PMCID: PMC5480083). Such toxicity is more difficult to manage if it results in the destruction of solid organs, the function of which may not be readily replaced.

Greater tumor specificity may be achieved by targeting epitopes derived from oncogenic viruses such as human papillomavirus (HPV). The HPV E6 and E7 oncoproteins are promising targets due to their exclusive expression in HPV-positive cervical, anal, head and neck cancers. However, cancers arising from infection with oncogenic viruses are a tiny minority of all diagnosed cancers and there exists a tremendous need for improvements to CAR T-cell targeting in the absence of unique tumor-associated antigens. Moreover, it has been well documented that cells within a tumor mass are typically heterogenous and unlikely to universally express a given tumor-associated antigen (TAA). When subjected to selective pressure driven by an antigen-specific immune response, further loss of TAA expression is likely to occur, rendering the therapy ineffective (Gerlinger M, Rowan A J, Horswell S, Math M, Larkin J, Endesfelder D, Gronroos E, Martinez P, Matthews N, Stewart A, Tarpey P, Varela I, Phillimore B, Begum S, McDonald N Q, Butler A, Jones D, Raine K, Latimer C, Santos C R, Nohadani M, Eklund A C, Spencer-Dene B, Clark G, Pickering L, Stamp G, Gore M, Szallasi Z, Downward J, Futreal P A, Swanton C. Intratumor heterogeneity and branched evolution revealed by multiregion sequencing. N Engl J Med. 2012 Mar. 8; 366(10):883-892. doi: 10.1056/NEJMoa1113205. Erratum in: N Engl J Med. 2012 Sep. 6; 367(10):976. PMID: 22397650; PMCID: PMC4878653).

Improvements to CAR T-cell therapy have been proposed that utilize an oncolytic virus, such as an engineered vaccinia virus, to infect tumor cells and express the CAR target antigen ectodomain (e.g., CD19) on the infected cell surface, with the drawback that few tumor cells are likely to be infected by the OV due to dosage titer constraints, immune-mediated virus clearance, and lack of suitable virus entry receptors among the multiple cell types present in the tumor (Park A K, Fong Y, Kim S I, Yang J, Murad J P, Lu J, Jeang B, Chang W C, Chen N G, Thomas S H, Forman S J, Priceman S J. Effective combination immunotherapy using oncolytic viruses to deliver CAR targets to solid tumors. Sci Transl Med. 2020 Sep. 2; 12(559):eaaz1863. doi: 10.1126/scitranslmed.aaz1863. PMID: 32878978; PMCID: PMC9126033) (AalipourA, Le Boeuf F, Tang M, Murty S, Simonetta F, Lozano A X, Shaffer T M, Bell J C, Gambhir S S. Viral Delivery of CAR Targets to Solid Tumors Enables Effective Cell Therapy. Mol Ther Oncolytics. 2020 Apr. 7; 17:232-240. doi: 10.1016/j.omto.2020.03.018. PMID: 32346612; PMCID: PMC7183102).

Despite advances in the art, current strategies do not address the fundamental problem of tumor heterogeneity and immune evasion through loss of targeted surface antigens.

The present invention overcomes shortcomings of current CAR T-cell therapies, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention provides compositions and methods for treating cancer by enhancing immune cell-based therapies (e.g., CAR T-cell therapy, CAR NK-cell therapy, CAR macrophage therapy, CAR neutrophil therapy or TCR-T cell immunotherapy) or antibody-drug conjugate (ADC) therapies with recombinant oncolytic viruses (OVs) that direct the expression of a soluble multivalent adapter protein (also referred to as an adapter protein, a soluble adapter protein or a soluble multivalent engager protein). Within various embodiments of the invention the multivalent proteins disclosed herein can be, for example, bivalent or bispecific, trivalent or trispecific, quadrivalent or quadraspecific, and/or pentavalent or pentaspecific.

As an alternative or supplement to OV-based delivery, the adapter protein may be delivered intratumorally either by direct injection of the purified adapter protein, or by injecting RNA encoding the adapter (as LNP-encapsulated mRNA, self-amplifying RNA, etc.). The multivalent adapter protein includes a first domain that is recognized by the chimeric antigen receptor, modified TCR, or ADC, and, in some embodiments, includes the ectodomain of the membrane protein the CAR, modified TCR, or ADC is designed to target (i.e., a first tumor-associated antigen). The multivalent adapter protein also includes a second domain that targets a tumor-associated cell surface antigen that is widely expressed and ubiquitous across multiple tumor types (i.e., a second tumor-associated antigen). In this manner, the CAR, modified TCR, or ADC is redirected from its original target that may have limited tumor expression to a broader array of tumor cells.

Within one embodiment of the invention, the first tumor-associated antigen recognized by the CAR, modified TCR, or ADC is an antigen overexpressed in solid tumors. Within a preferred embodiment, the first tumor-associated antigen is selected from the group consisting of CD19, BCMA, HER2, EGFR, IL13Ra2, CEA, EGFRvIII, Mesothelin, Claudin 18.2, PSCA, CAIX, AXL, PSMA, Folate receptor-alpha, MUC16, MUC1, ROR1, and OR2H1. In other embodiments, the second tumor-associated antigen recognized by the multivalent adapter protein is expressed across a broader range of tissues or cell types compared to the first tumor-associated antigen. In other preferred embodiments, the second tumor-associated antigen recognized by the multivalent adapter protein is selected from the group consisting of TfR1 and GLUT1.

Within yet another embodiment, the first domain of the multivalent adapter protein that is recognized by the chimeric antigen receptor or modified T cell receptor is located near the 5′ end of the coding region for the multivalent adapter protein and the second domain of the multivalent adapter protein that targets a tumor-associated cell surface antigen that is widely expressed and ubiquitous across multiple tumor types is located near the 3′ end of the coding region for the multivalent adapter protein, with the two domains separated by a linker. Alternatively, the first domain of the multivalent adapter protein that is recognized by the chimeric antigen receptor or modified T cell receptor is located near the 3′ end of the coding region for the multivalent adapter protein and the second domain of the multivalent adapter protein that targets a tumor-associated cell surface antigen that is widely expressed and ubiquitous across multiple tumor types is located near the 5′ end of the coding region for the multivalent adapter protein, with the two domains separated by a linker.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to Improve drawing legibility. The particular shapes of the elements as drawn have been selected for ease of recognition in the drawings. One or more embodiments are described hereinafter with reference to the accompanying drawings in which:

FIG. 1 illustrates one embodiment of using an OV-delivered secretable multivalent adapter protein to tag cells for immune destruction.

FIGS. 2A, 2B, 2C and 2D are bar graphs depicting cellular cytotoxicity, CD69 upregulation, expression of human granzyme B, and expression of human INF-g, respectively, in tumor cells treated with the multivalent adapter protein CD19-H7 in the presence of CAR-T cells engineered to target CD19.

FIGS. 3A, 3B, 3C and 3D are bargraphs depicting differences in CD69 expression by αHER2-CAR-Jurkat cells exposed to different multivalent engager molecules in the presence of HER2⁺ A549 cells, HER2⁺ MCF7 cells, HER2⁻ 293FT cells, and HER2⁻ MDA-MB-231 cells, respectively.

FIGS. 4A to 4Z and 4AA to 4WW are a list of tumor antigens, setting forth the antigen name, common name, as well as synonyms.

FIGS. 5A, 5B, 5C, and 5D illustrate the effect of gD-H7 decoration on induction of CAR T-cell function in A549 cells as measured by cytotoxicity (FIG. 5A), CD69 expression (FIG. 5B), IL-2 production (FIG. 5C), and Granzyme B production (FIG. 5D).

FIG. 6A is a bar graph which depicts the cytotoxicity of VG22001 expressing CD19-αTfR and VG2003 without CD19-αTfR in A549 cells, when co-cultured with CD19-CAR-T cells or Mock T cells.

FIG. 6B is a bar graph which depicts the IFN-γ levels in the supernatant of A549 cells infected with VG22001 expressing CD19-αTfR and VG2003 without CD19-αTfR, when co-cultured with CD19-CAR-T cells or Mock T cells.

FIG. 7 illustrates one embodiment of using an intratumorally injected or OV-delivered secretable adapter protein to enable a therapeutic antibody drug conjugate (ADC) to recognize and enter cells that fail to express, or have low expression of, the ADC target antigen on the cell surface.

FIGS. 8A and 8B are bar graphs depicting cytolysis at 48 hours post-treatment or at 72 hours post-treatment, respectively, in the HER2 low-expression cell line MDA-MB-468 after treatment with the adapter protein HER2-αTfR in the presence of the anti-HER2 ADC T-DM1. Error bars represent standard deviation.

FIGS. 9A and 9B are bar graphs depicting cell viability and apoptosis, respectively, in HER2-negative Raji cells treated with the adapter protein HER2-αTfR in the presence of the anti-HER2 ADCT-DM1. Error bars represent standard deviation.

### DETAILED DESCRIPTION OF THE INVENTION

The present invention may be understood more readily by reference to the following detailed description of preferred embodiments of the invention and the Examples included herein.

The term “T cell genetically modified to express a chimeric antigen receptor (CAR),” i.e., “CAR T-cell,” refers to a T cell transduced to express a chimeric antigen receptor (“CAR”). CARs are molecules that combine antibody-based specificity for a desired antigen (e.g., tumor-associated or tumor-specific antigen) with a T cell receptor-activating intracellular domain to generate a chimeric protein that exhibits a specific anti-tumor cellular immune activity. Alternatively, T cells can be isolated and genetically modified in vitro to express TCRs that target specific tumor antigens (TCR-T cell therapy). In some embodiments, CAR T cells are engineered to express antigen binding domains of a monoclonal antibody or antibody fragment, such as, for example, a Fab or an scFv.

The term “antibody” or “antibodies” refers to both full-length immunoglobulins (i.e., naturally occurring or recombinantly formed whole molecules) (e.g., an IgG antibody such as IgG1, IgG2a, IgG3, IgG4 (and IgG4 subforms), IgA isotypes, IgE and IgM) or an immunologically active (i.e., specifically binding) portion of an immunoglobulin molecule, such as an antibody fragment or segment. Representative antibody fragments or segments include separate heavy chains, light chains, and portions of an antibody such as F(ab′)₂, F(ab)₂, Fab′, Fab, Fv, scFv (single chain Fv) and the like, including the half-molecules of IgG4 (see van der Neut Kolfschoten et al. (Science 2007; 317 (14 September): 1554-1557). Antibody fragments or segments also include immunologically active minimal recognition units consisting of the amino acid residues that mimic the hypervariable region, such as CDRs. Antibody fragments or segments can be produced by enzymatic or chemical separation of intact immunoglobulins, or by recombinant techniques. The term “antibody” should also be understood to include one or more immunoglobulin chains that are chemically conjugated to, or expressed as fusion proteins along with other proteins.

The term “antibody” also includes single domain antibodies (sdAbs) and nanobodies. SdAbs are comprised of a single monomeric variable region and may be derived from either heavy chains or light chains. One advantage of sdAbs over traditional monoclonal antibodies or other antibodies such as scFv or diabodies is the significantly smaller size (^{˜}2 nm) of single domain antibodies, thus making them less likely to interfere with the functions of essential glycoproteins on the viral envelope due to steric hindrance. In addition, sdAbs and nanobodies exhibit high affinity towards their targets and excellent biophysical properties such as thermal stability.

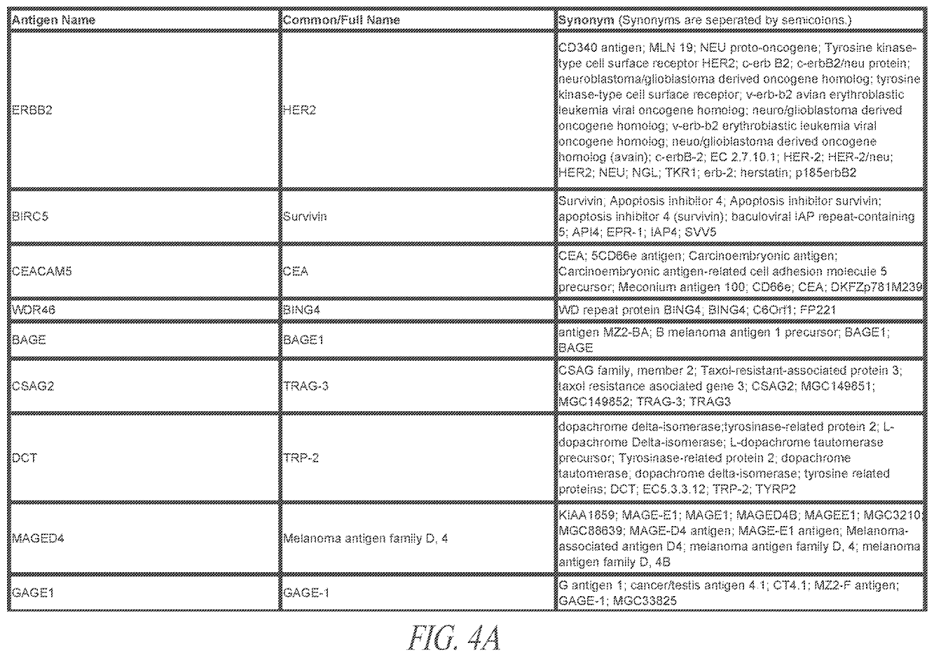

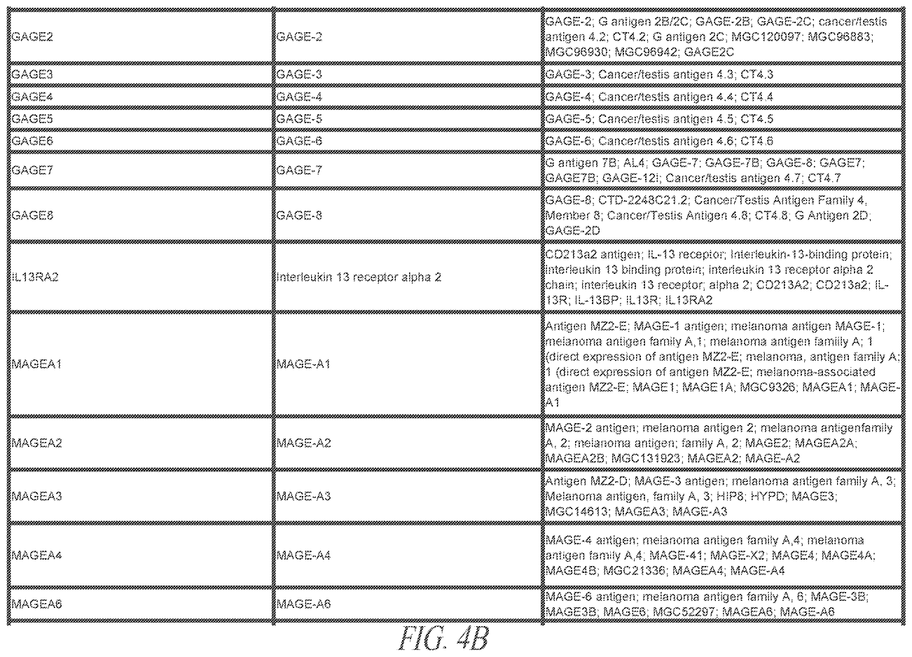

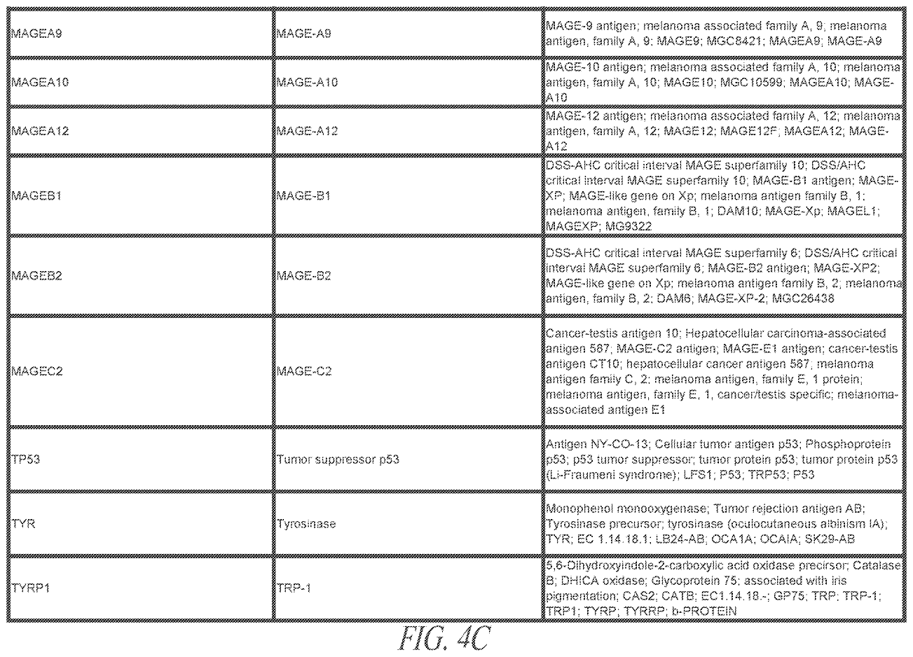

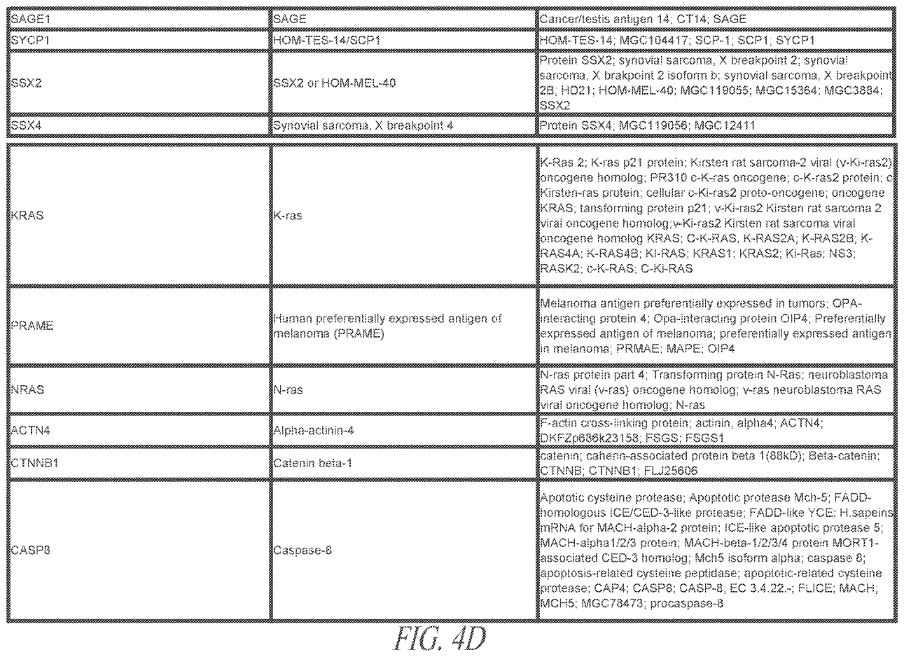

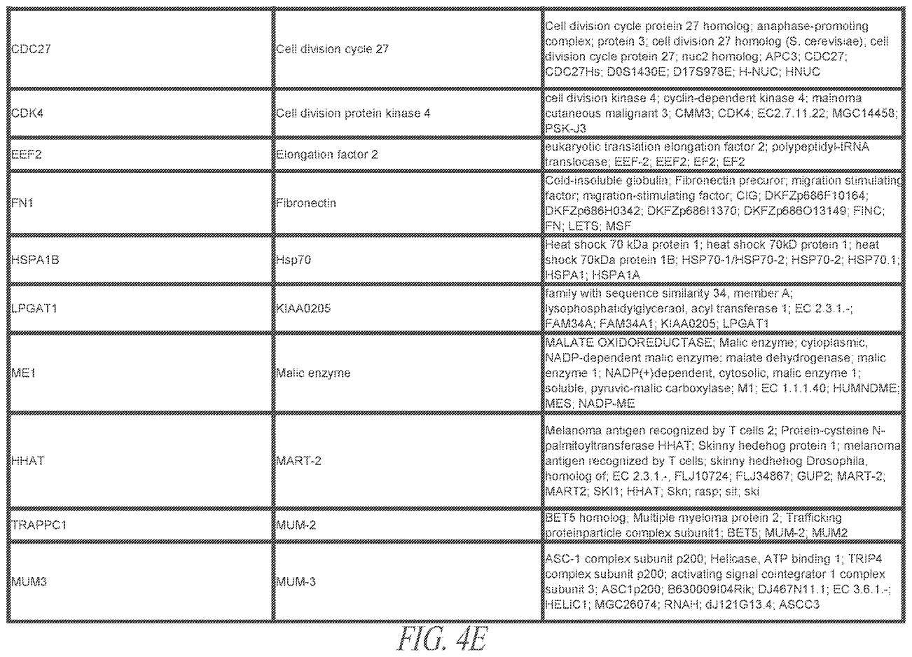

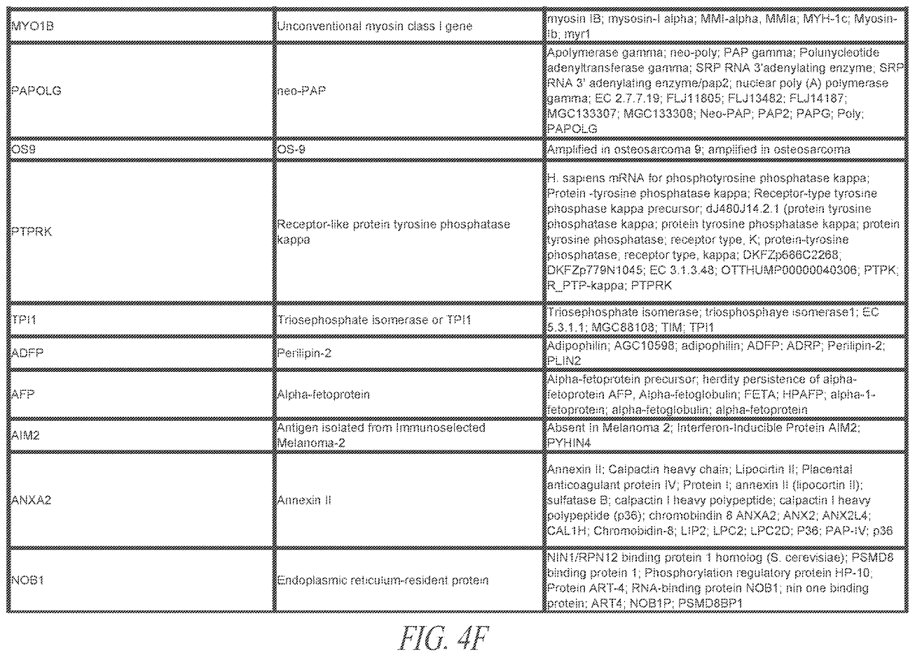

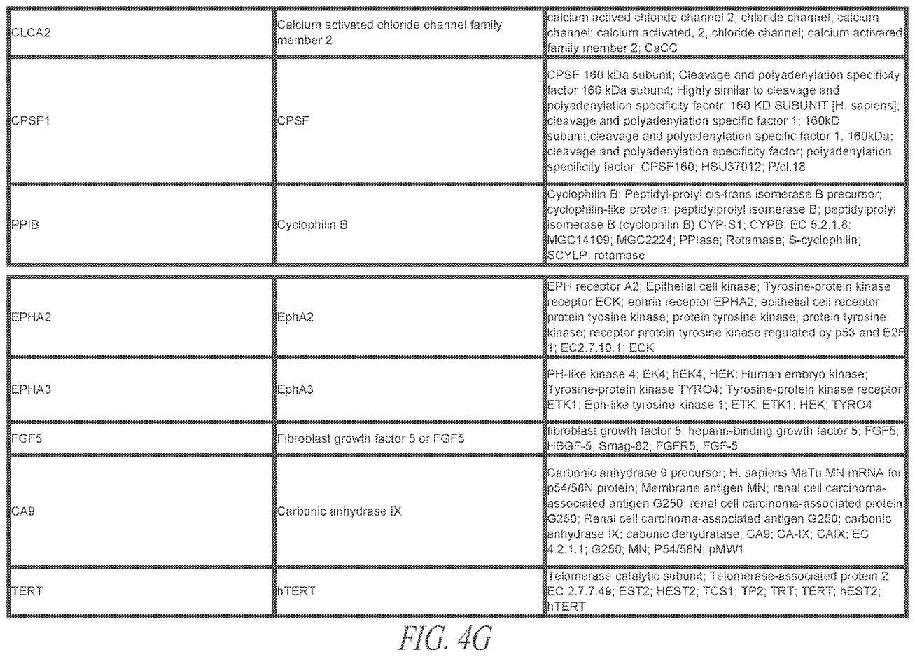

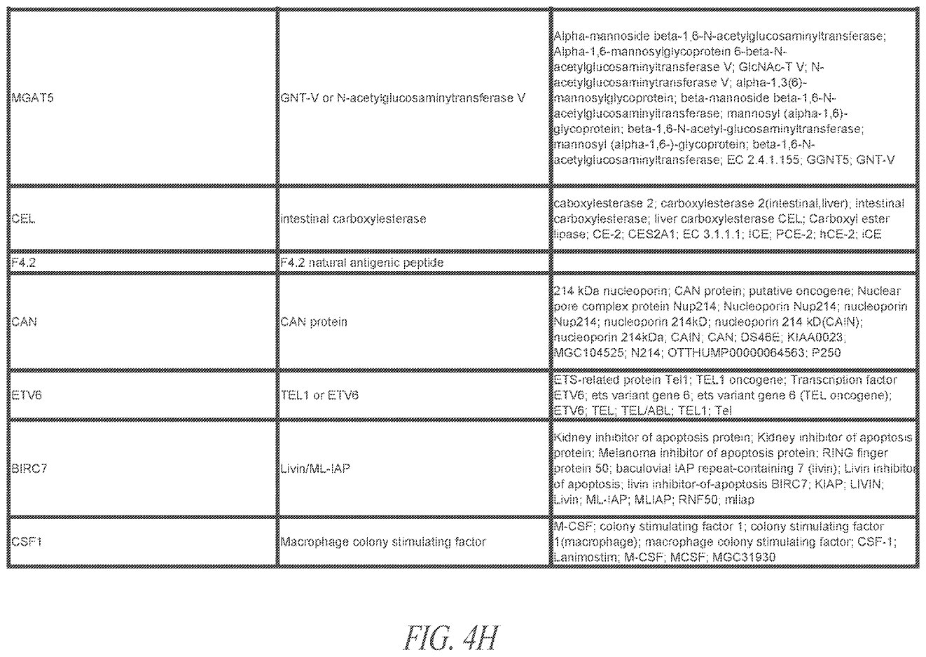

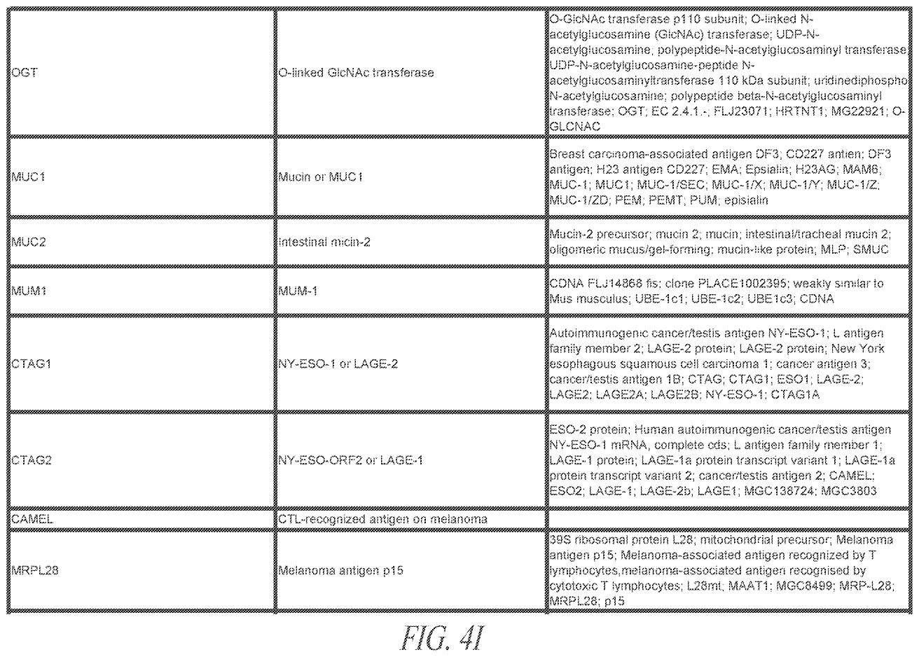

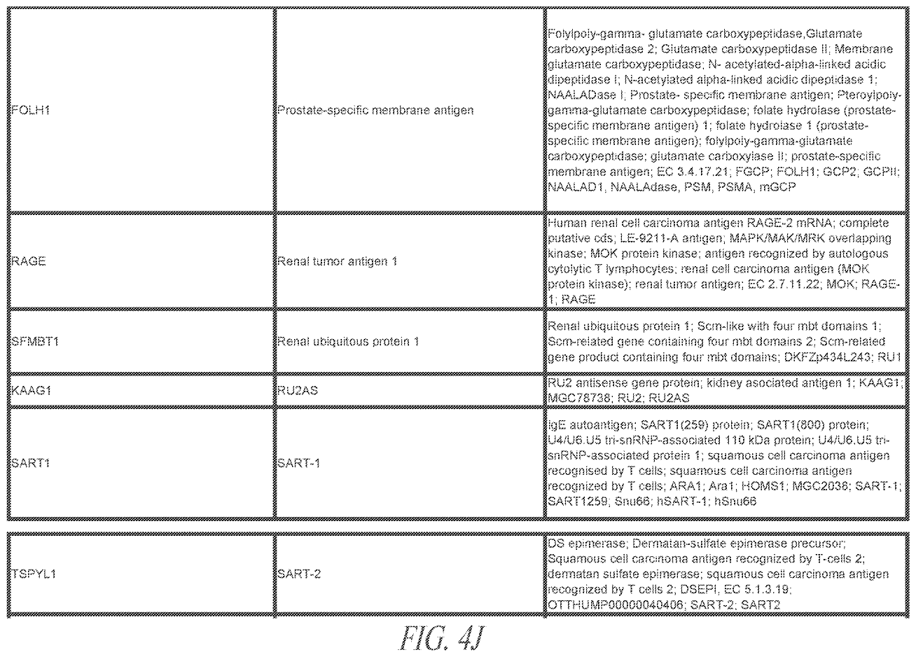

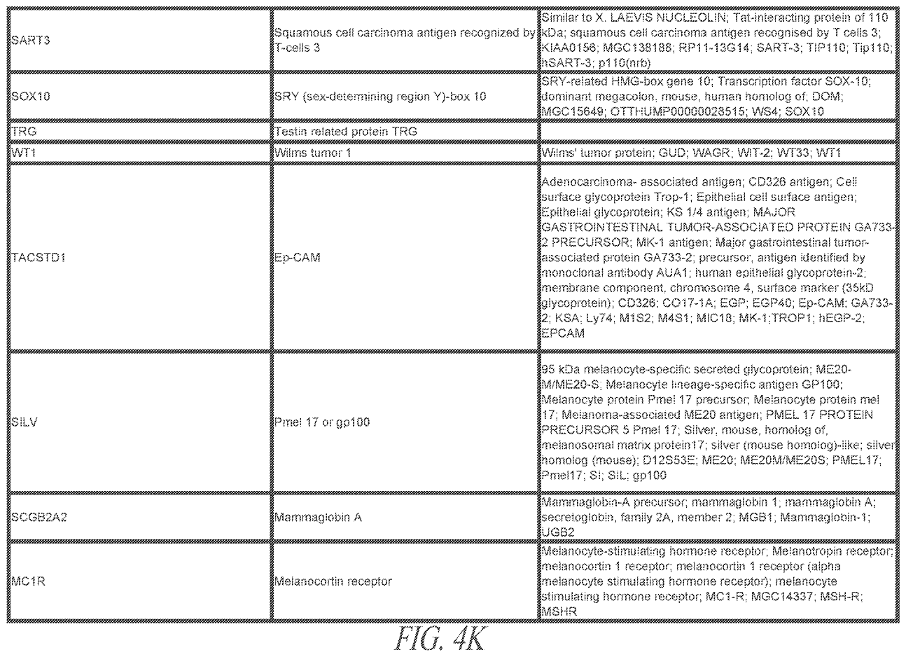

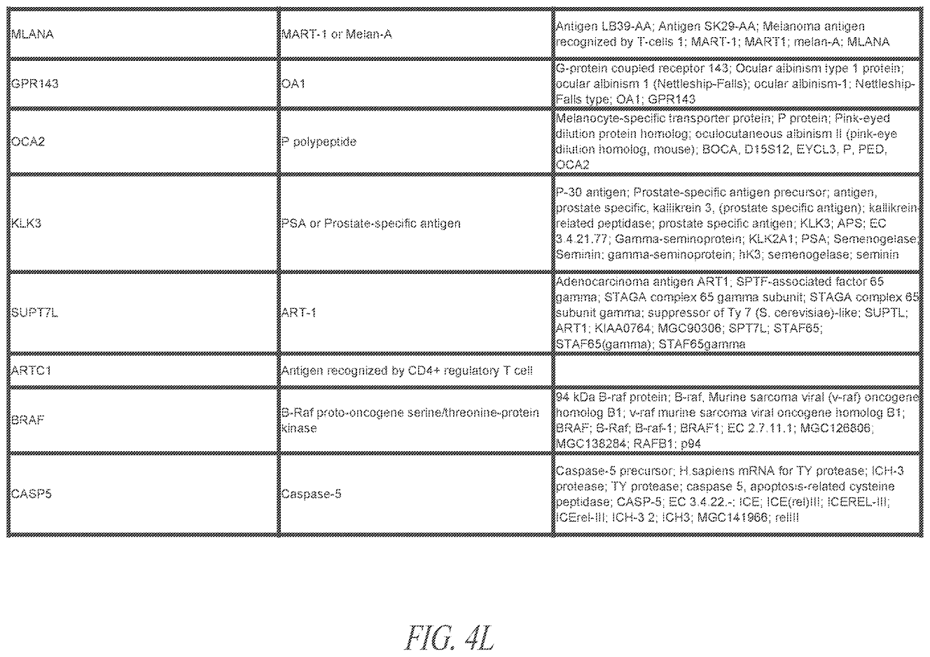

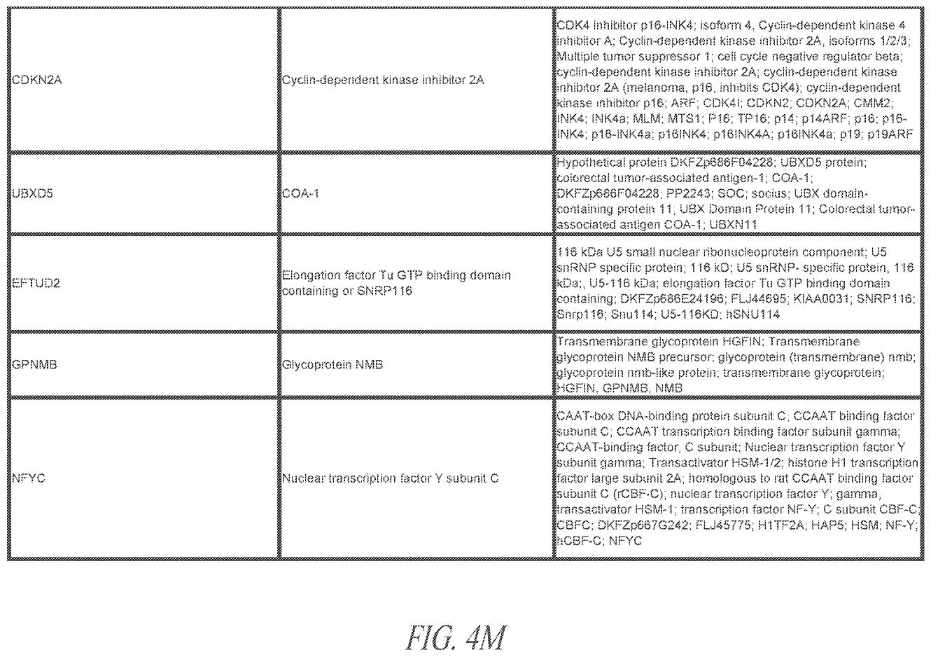

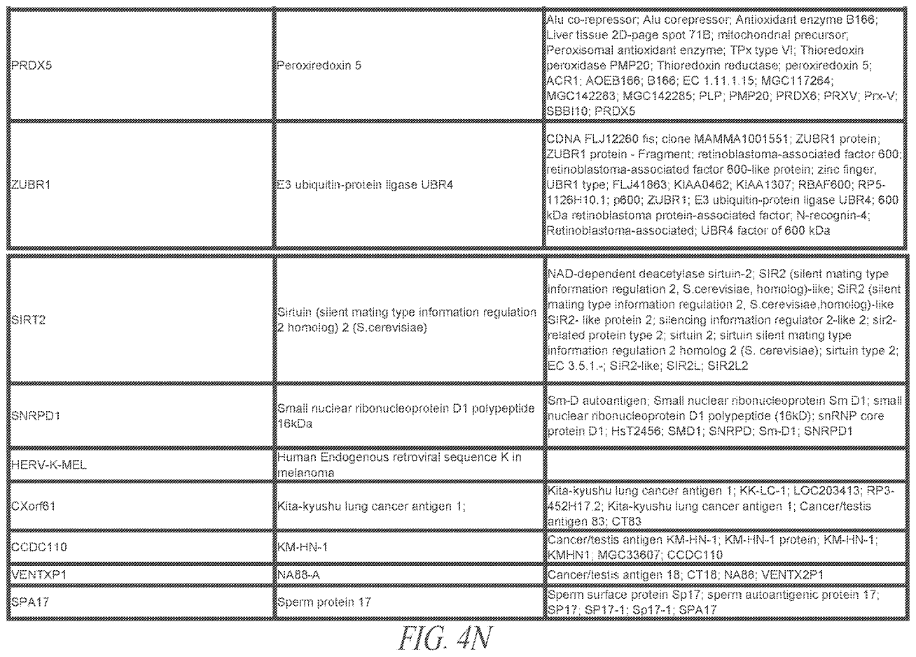

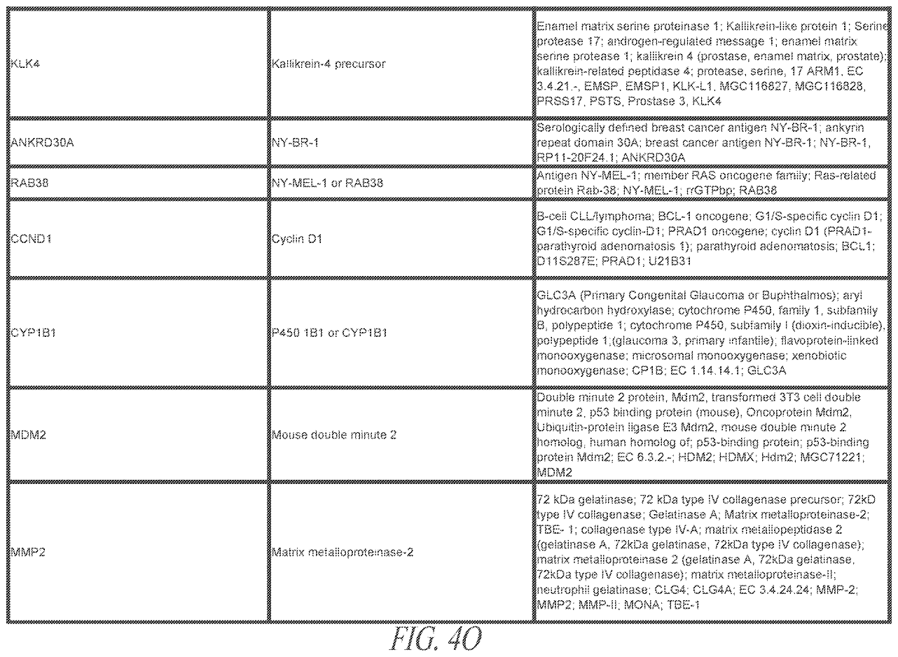

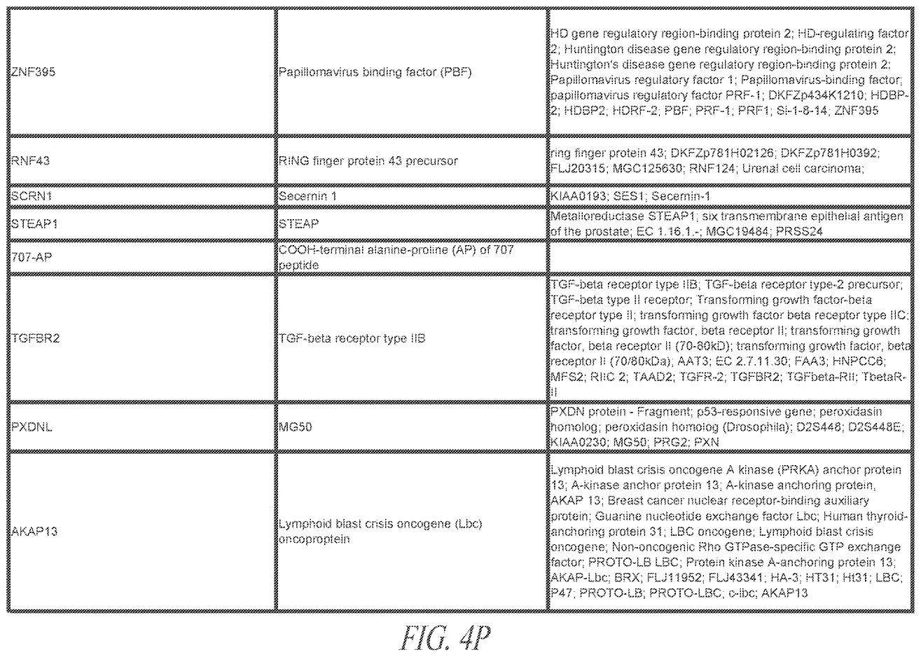

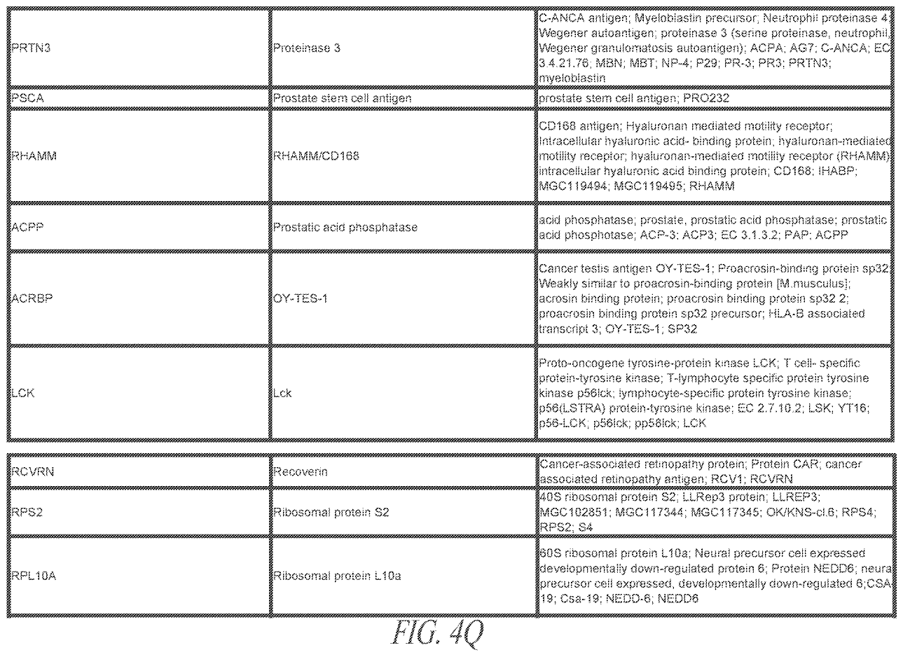

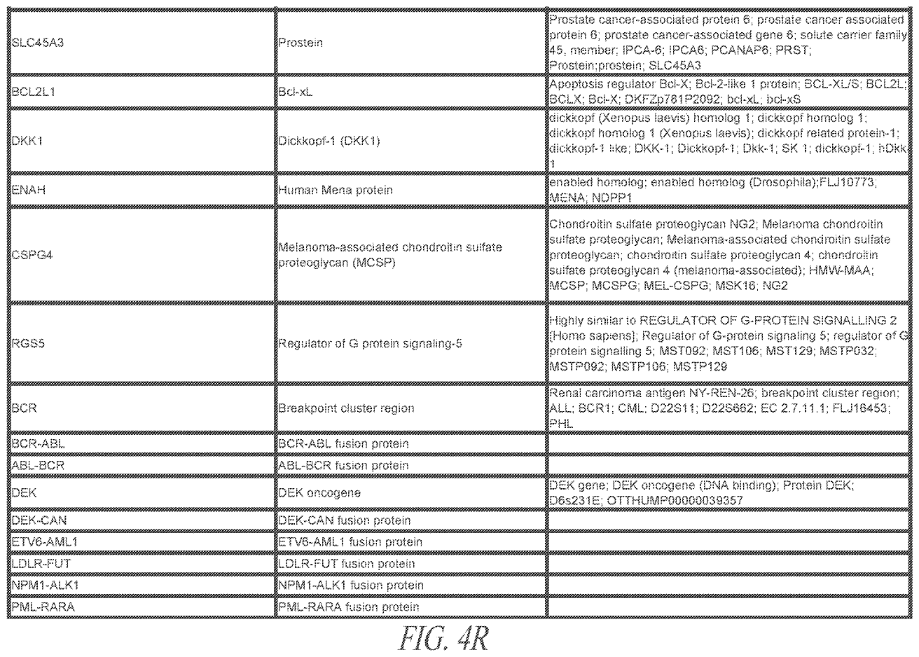

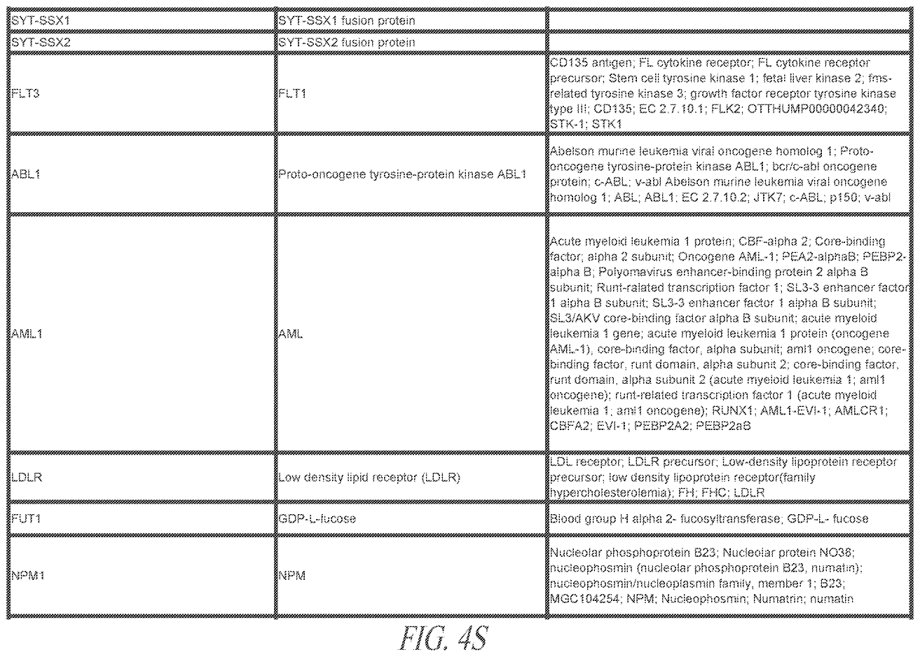

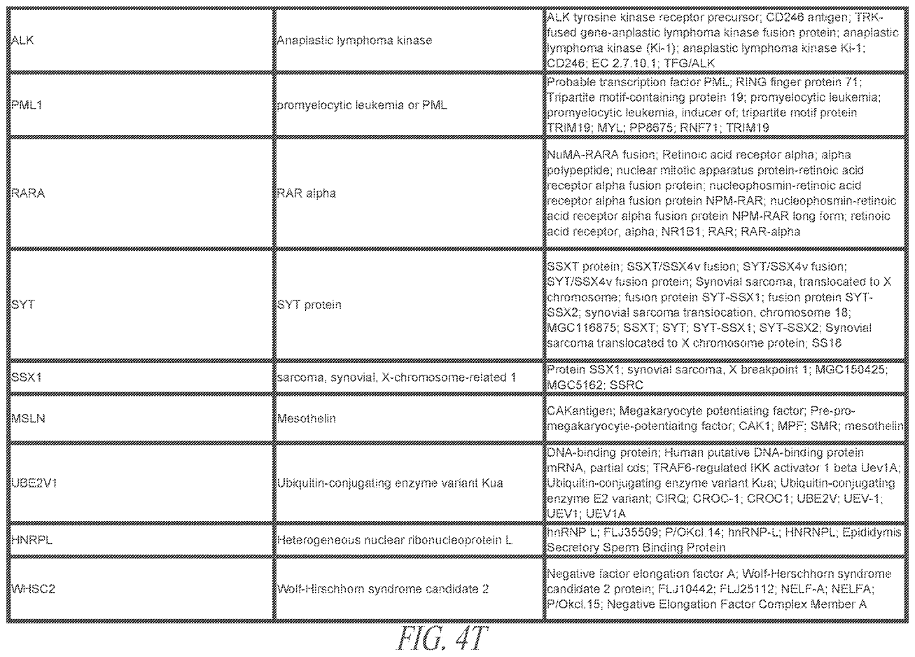

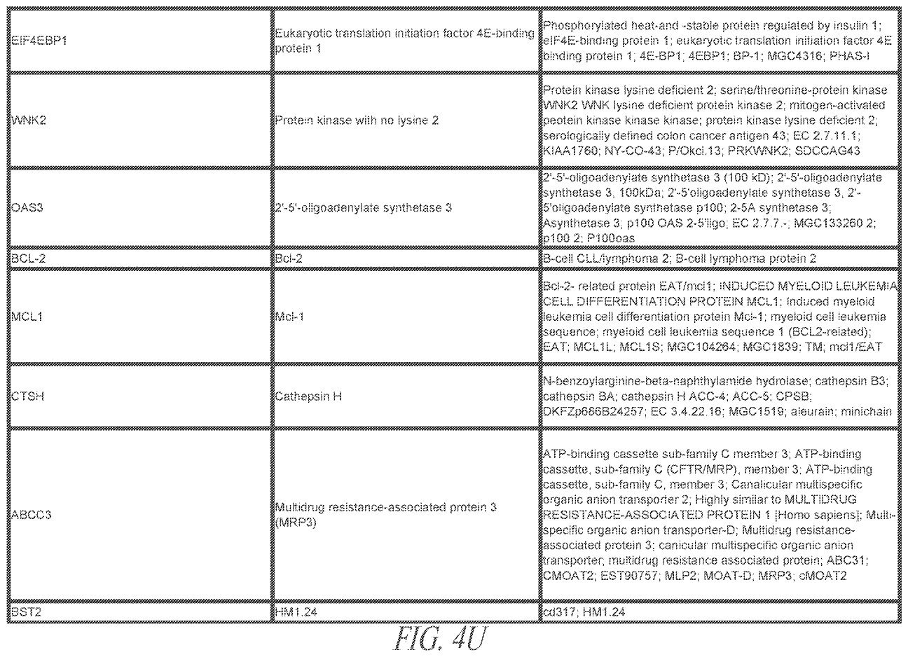

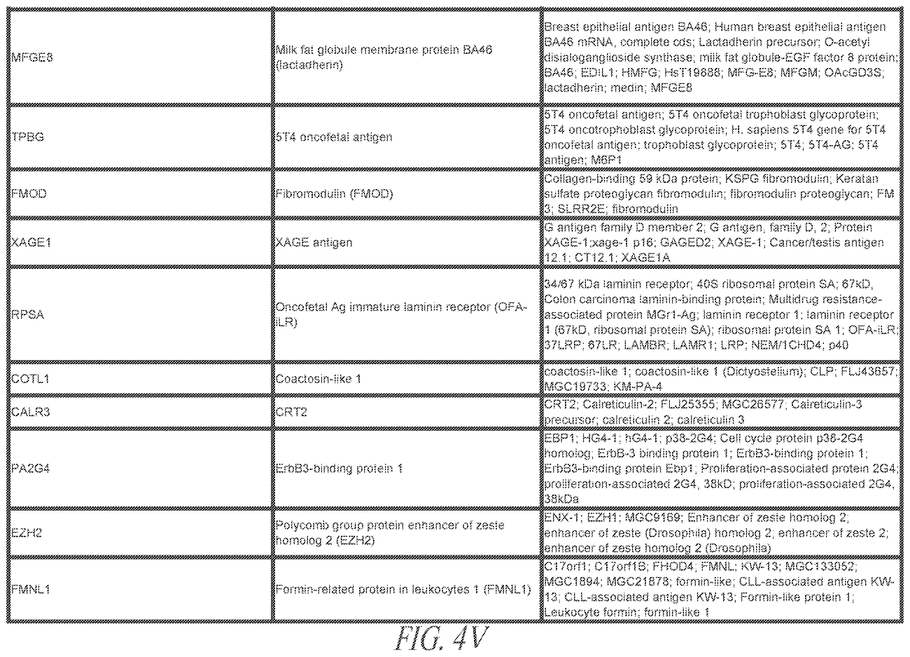

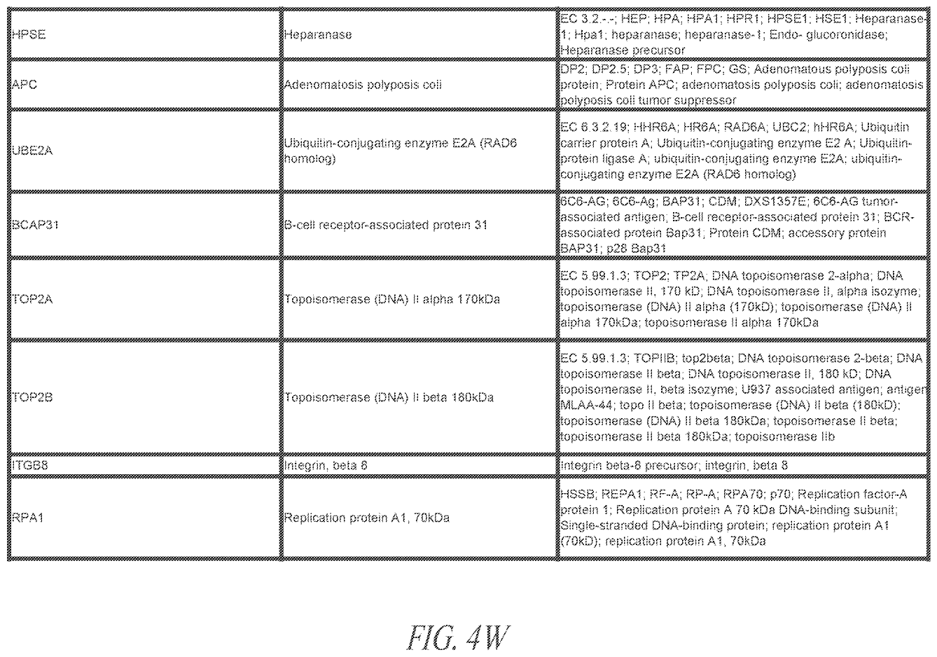

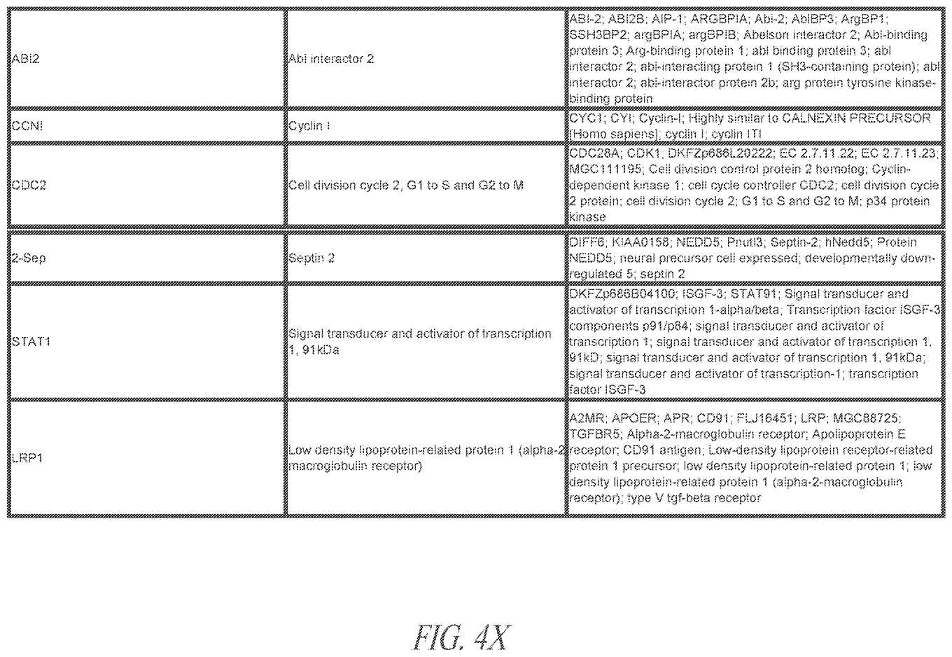

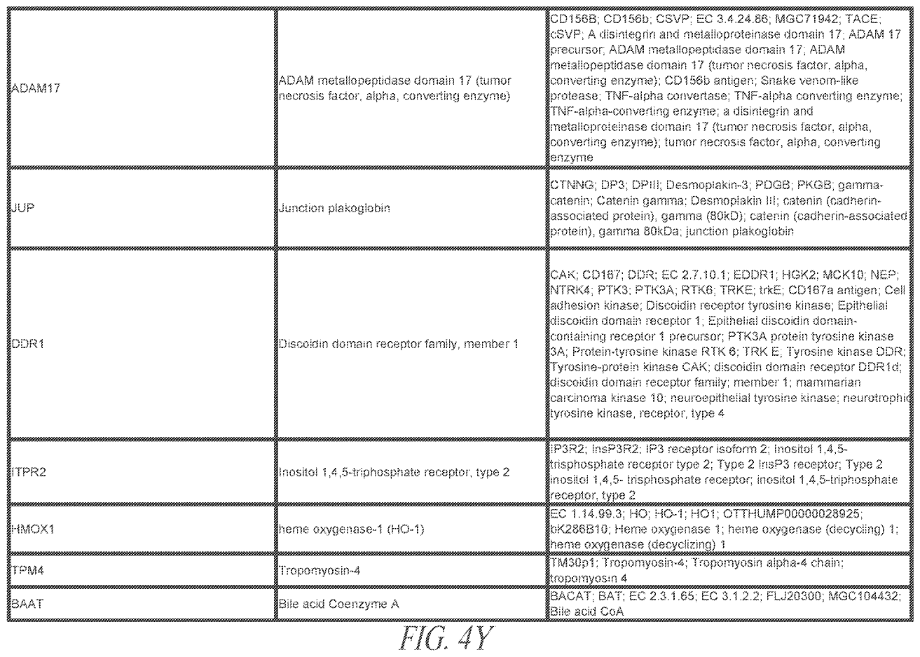

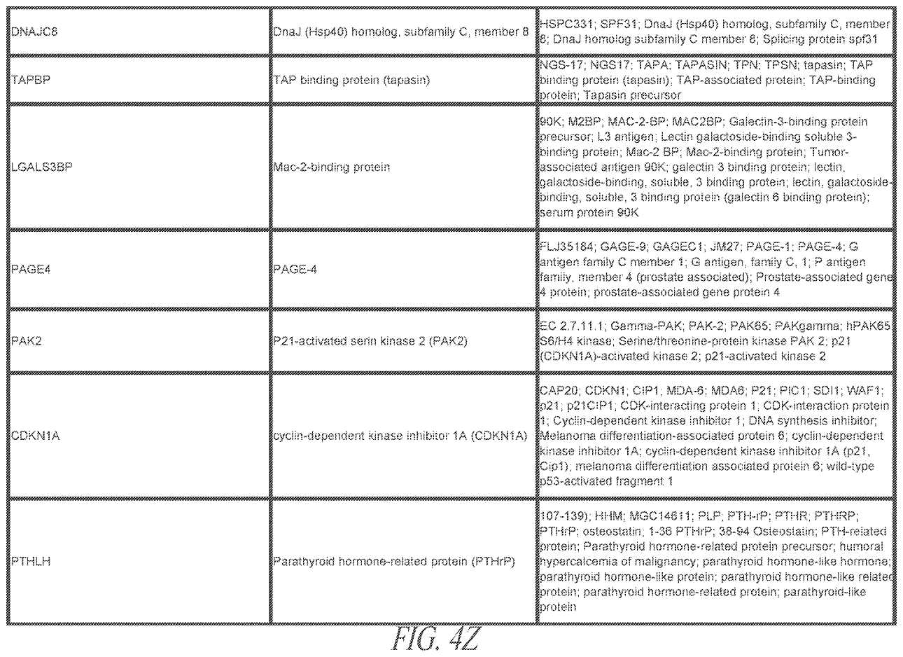

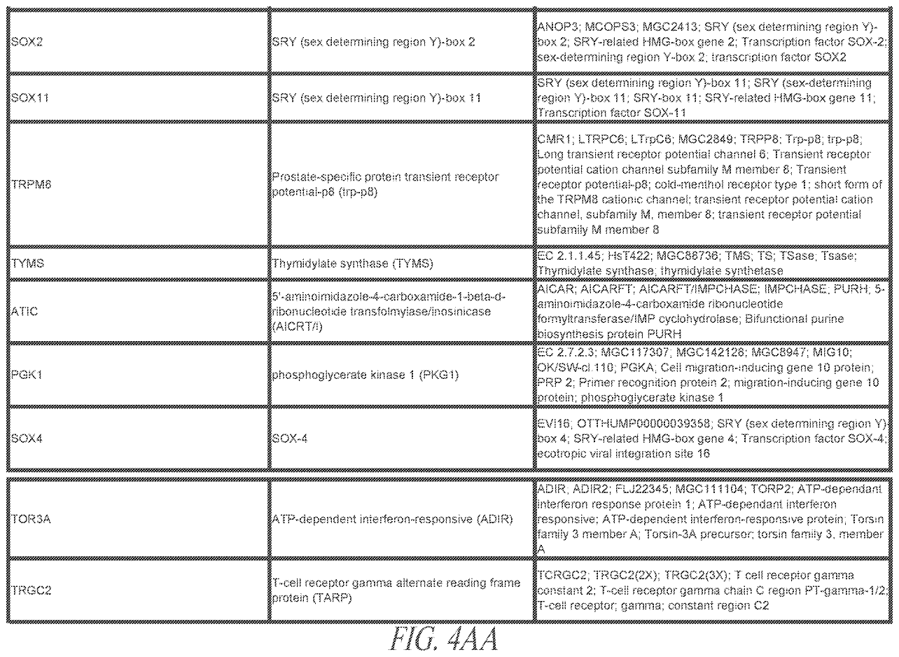

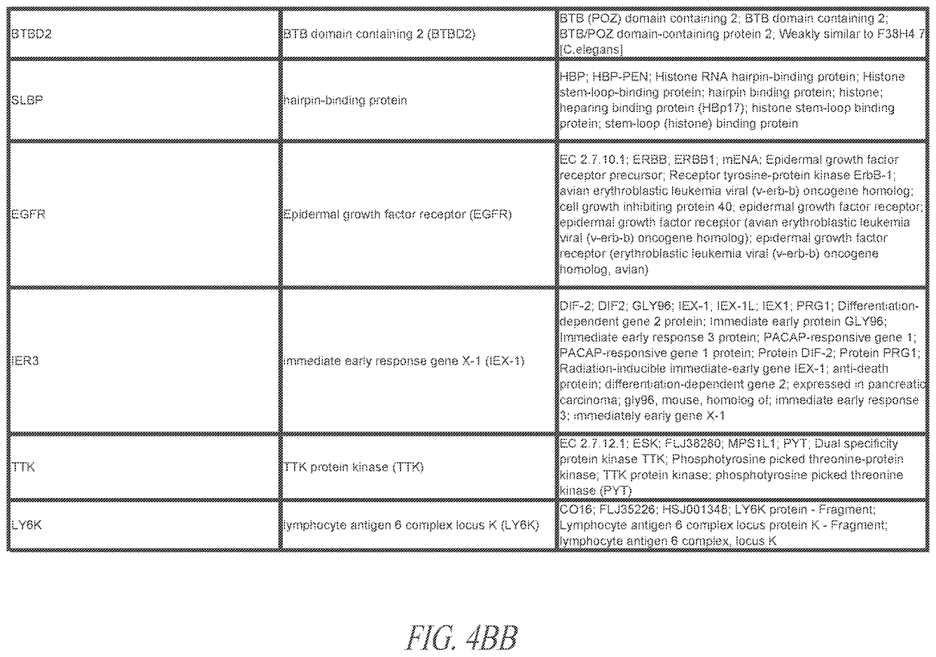

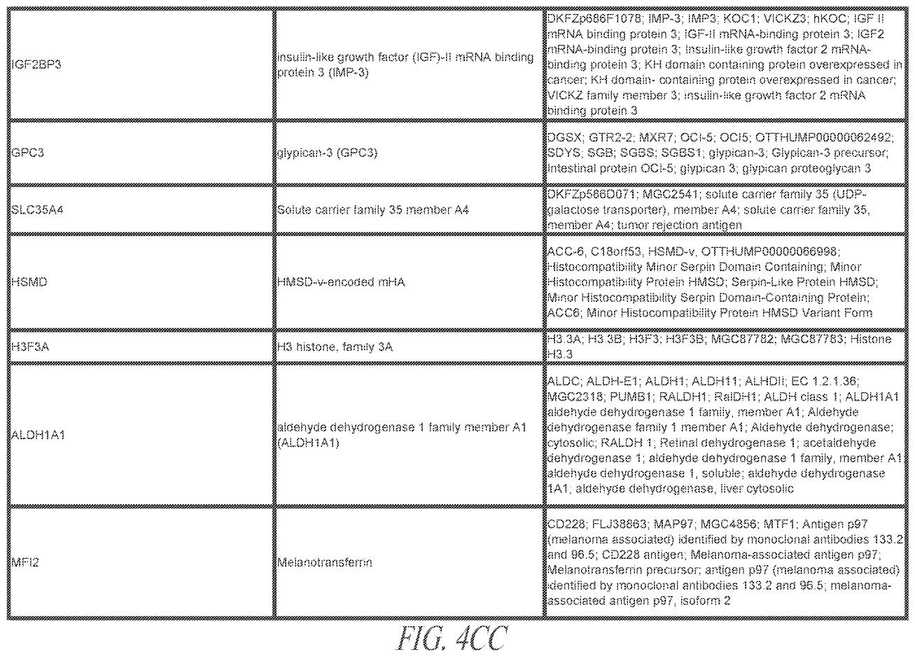

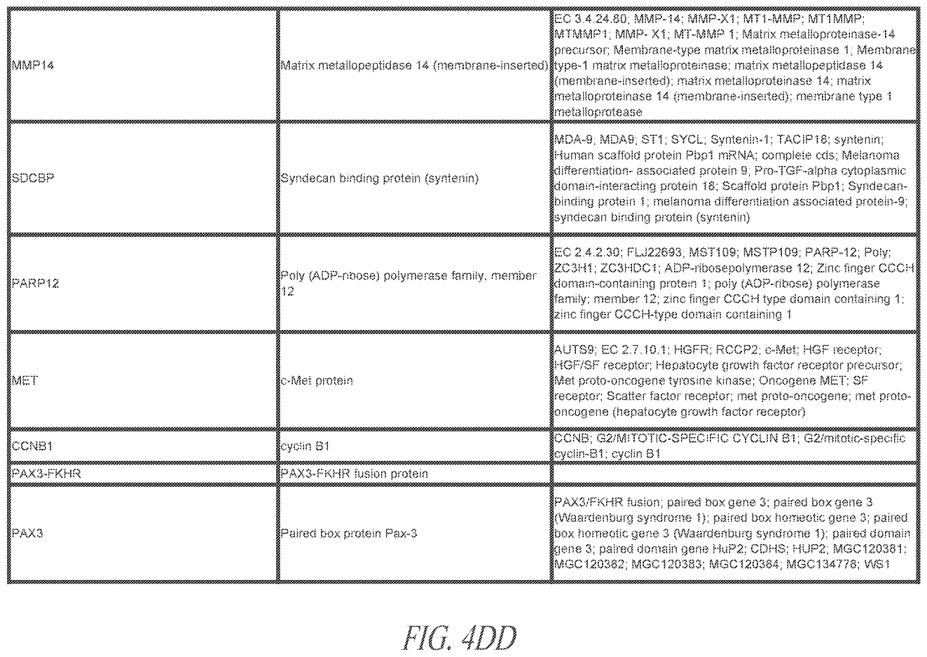

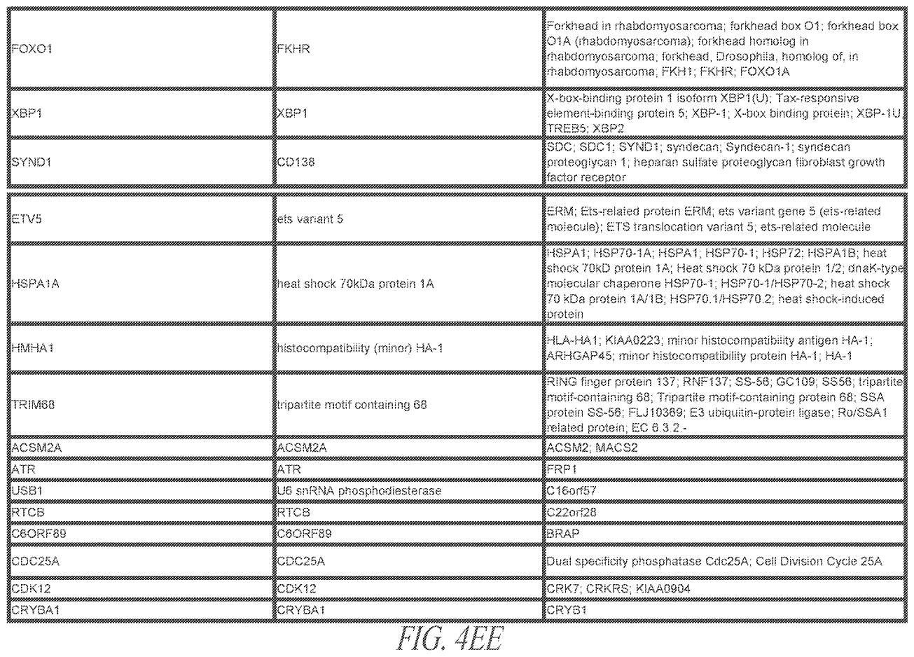

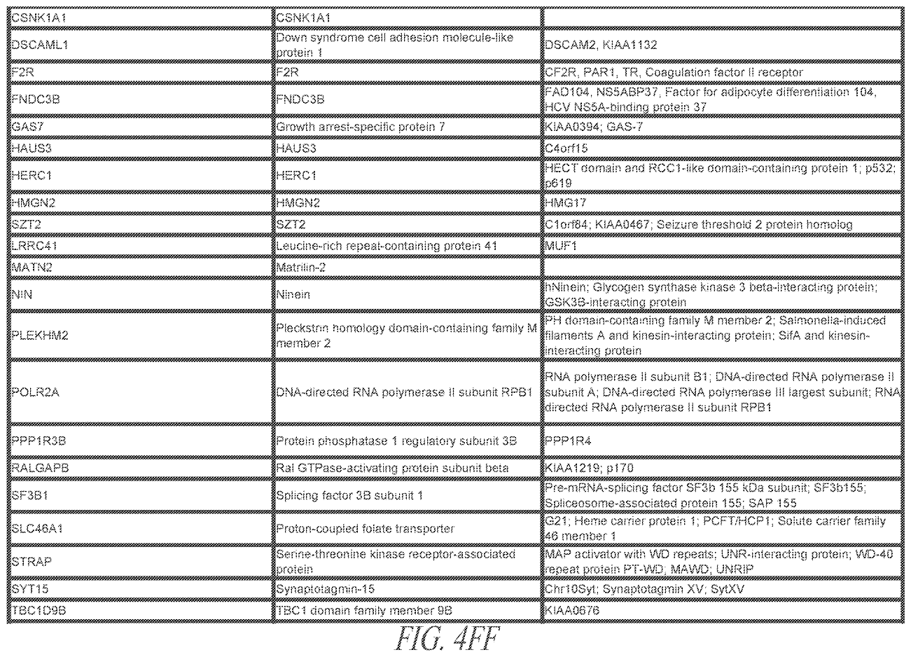

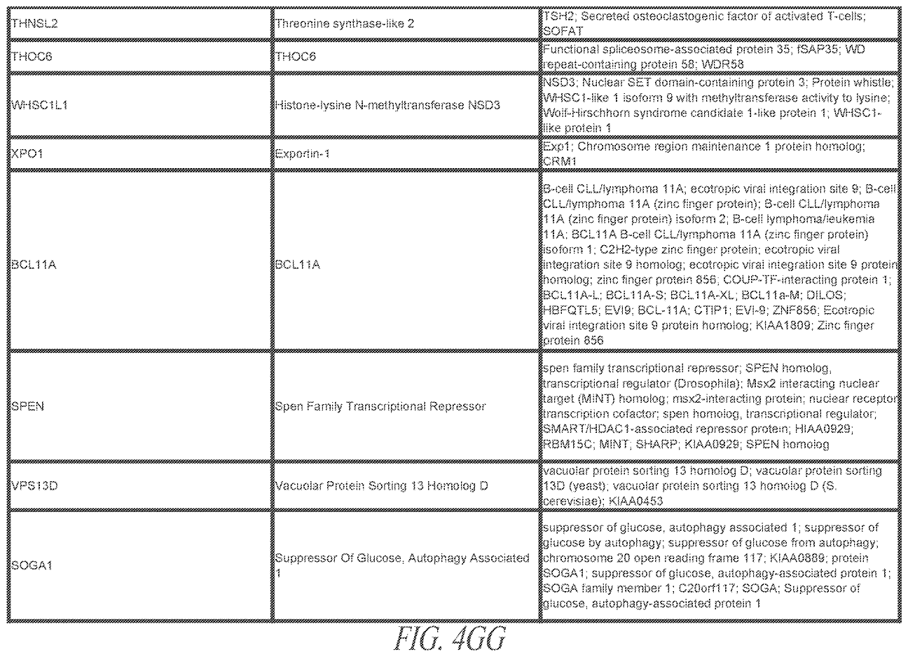

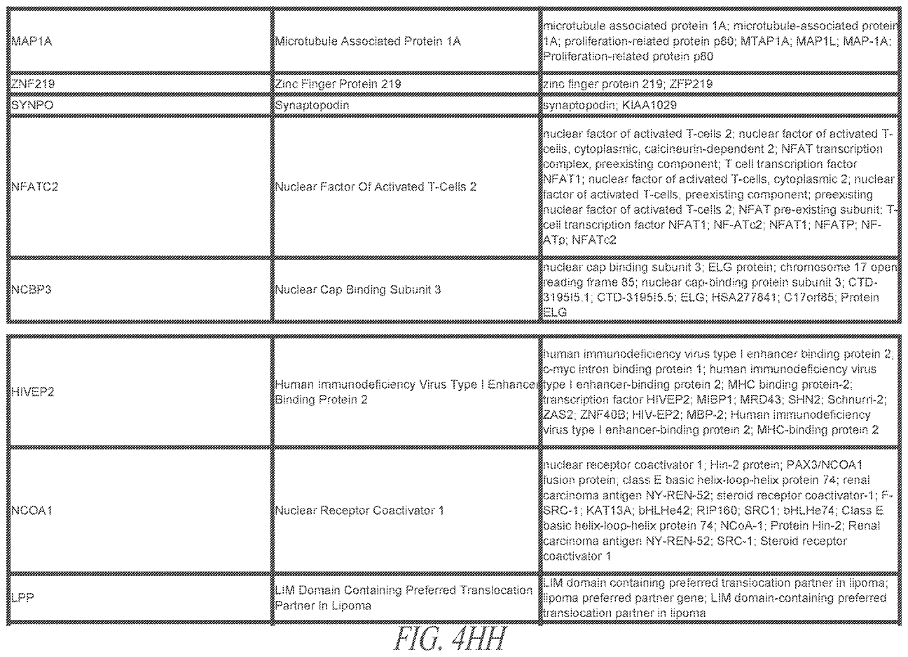

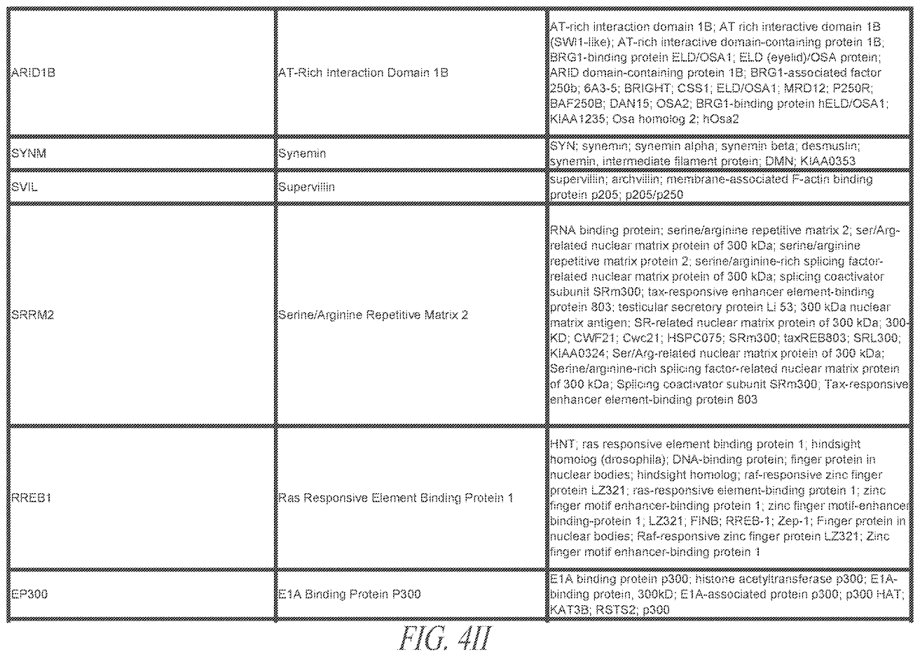

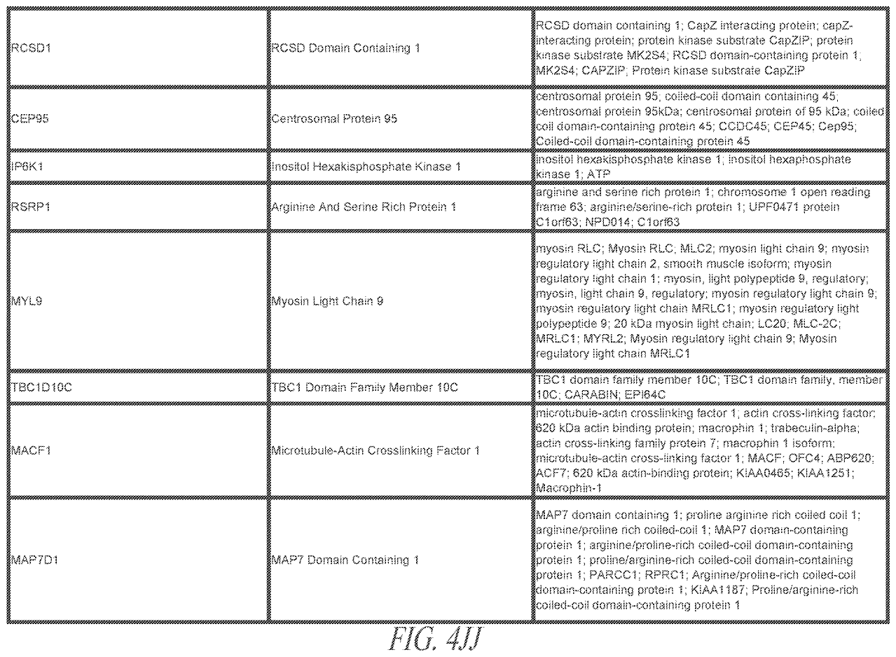

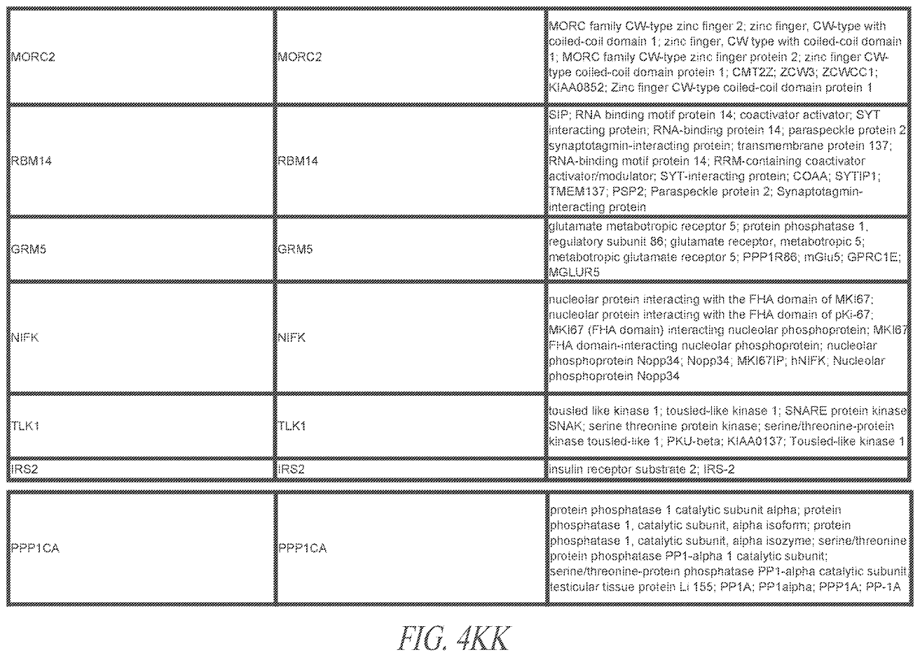

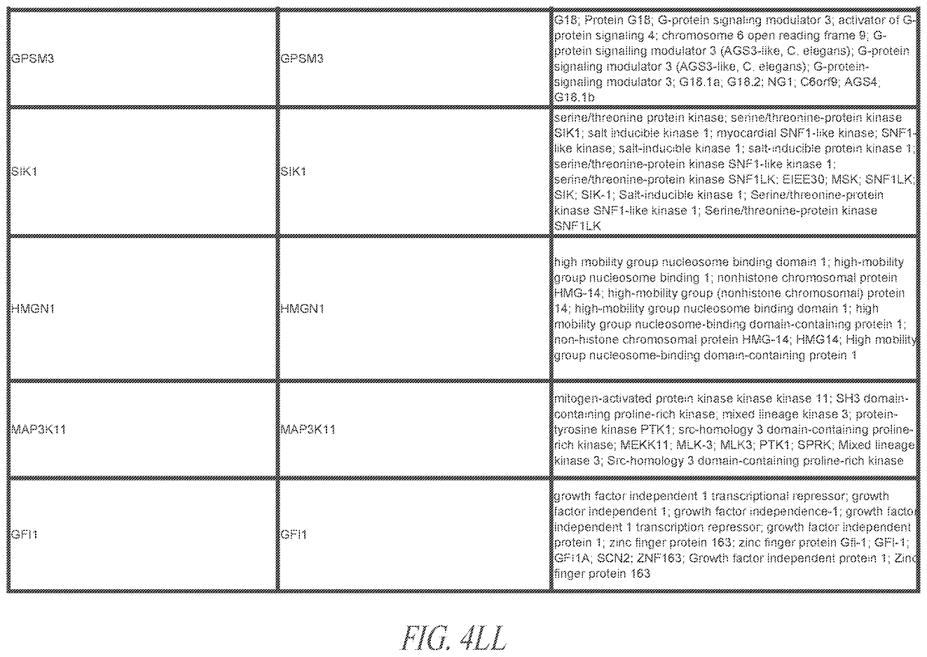

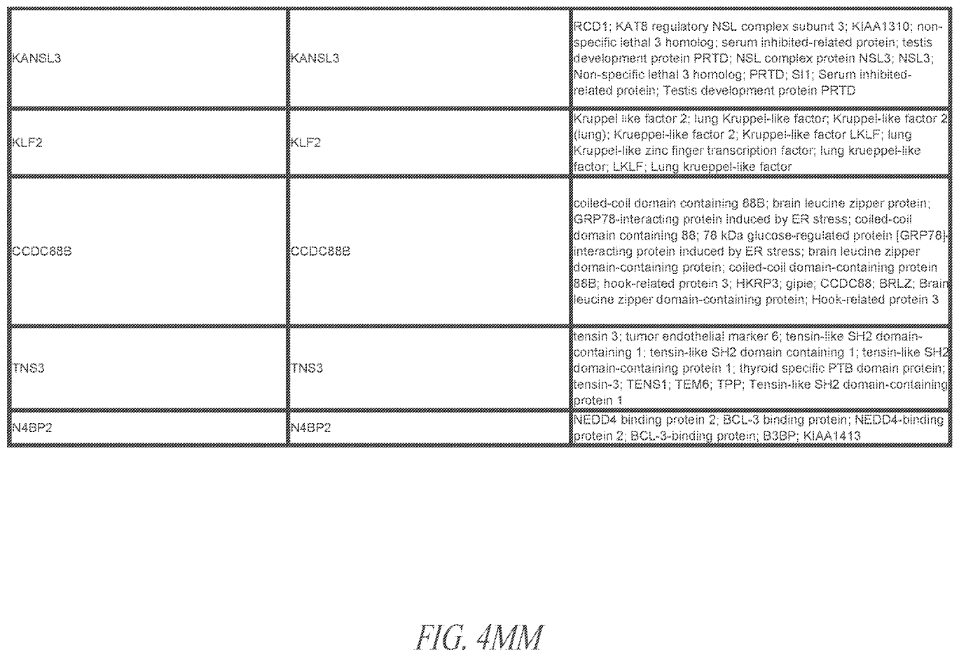

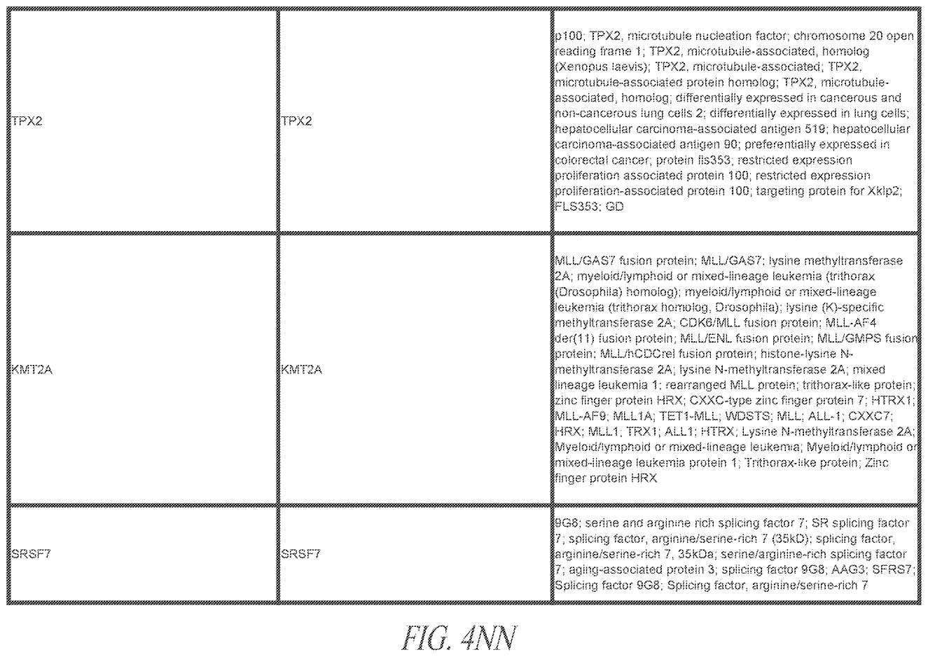

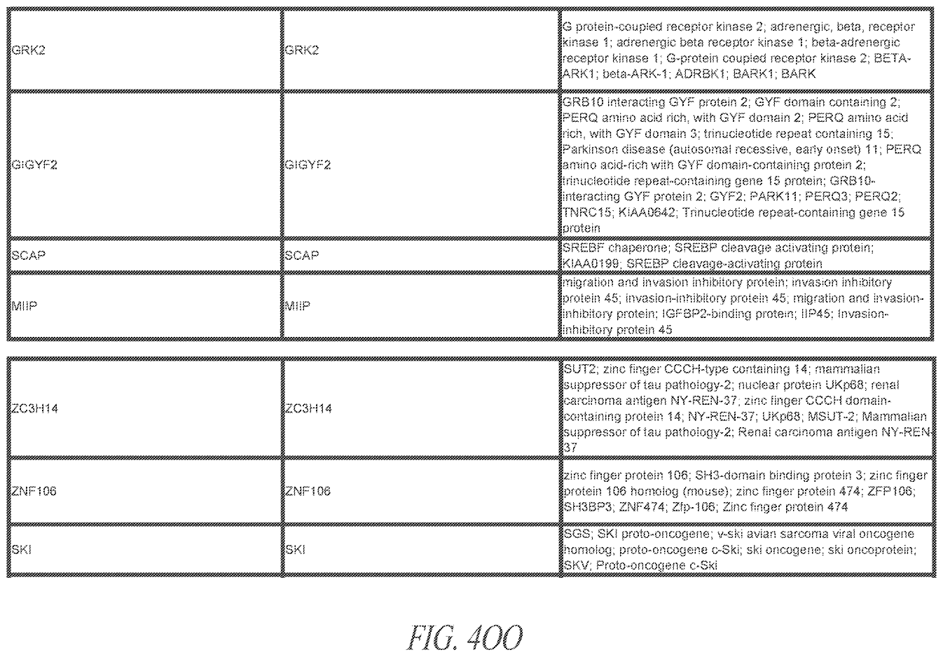

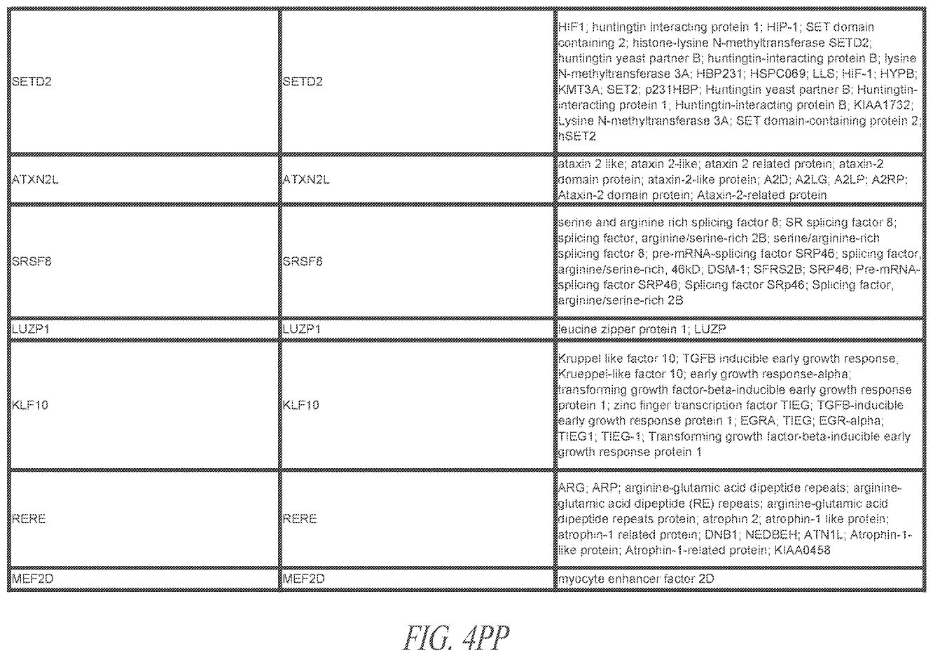

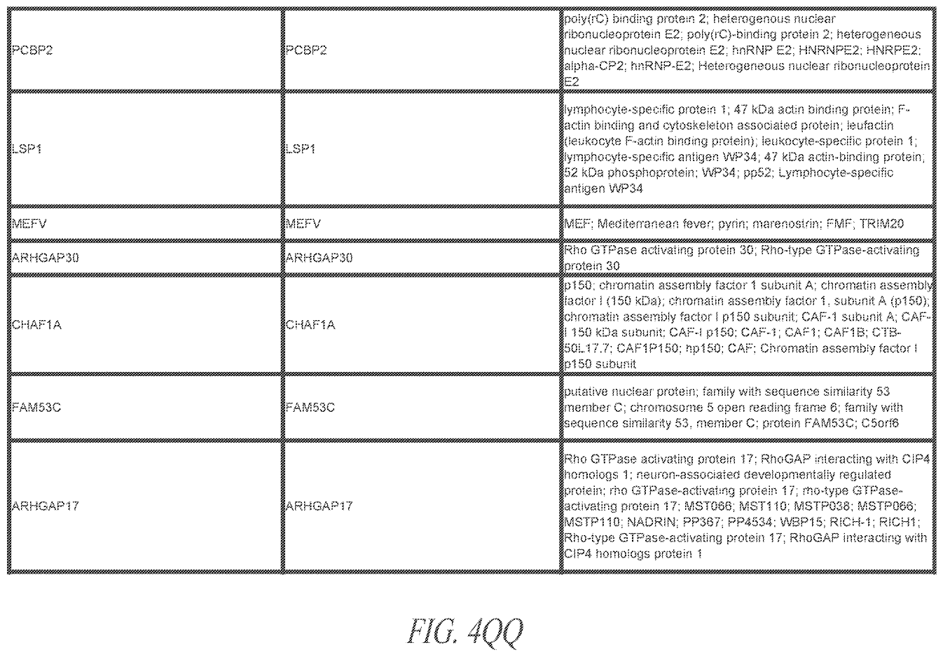

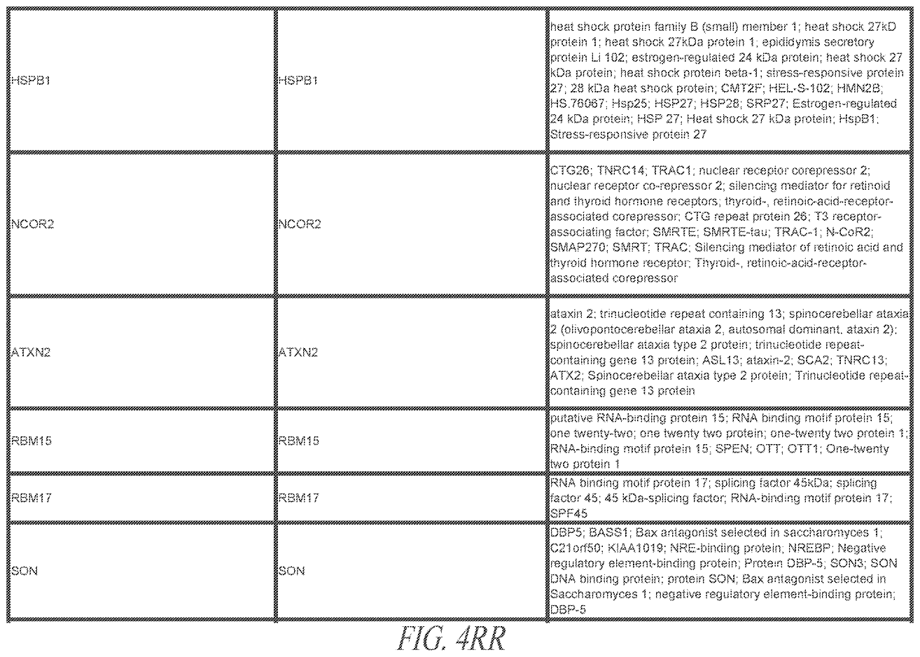

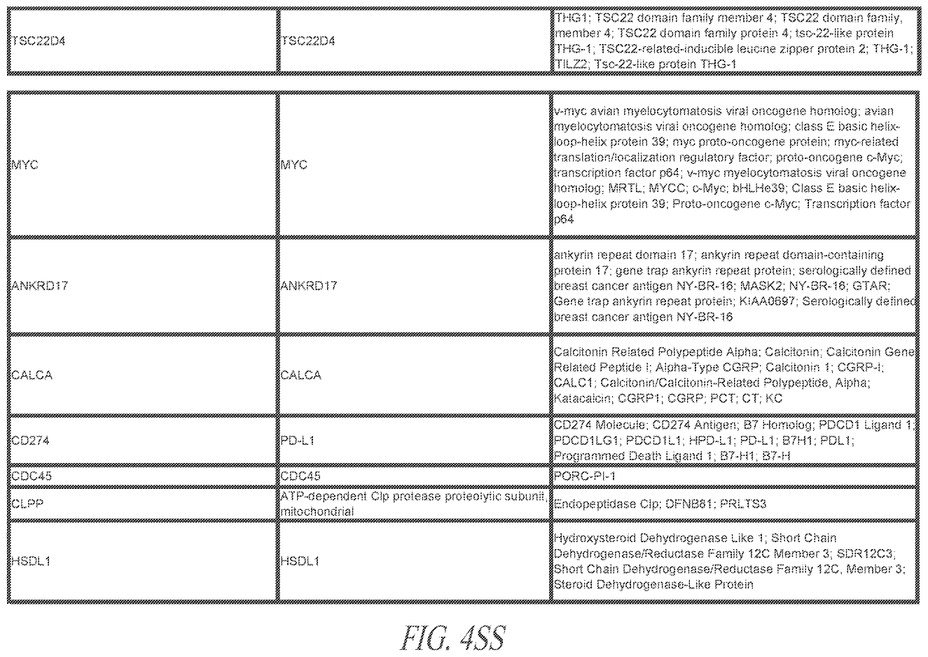

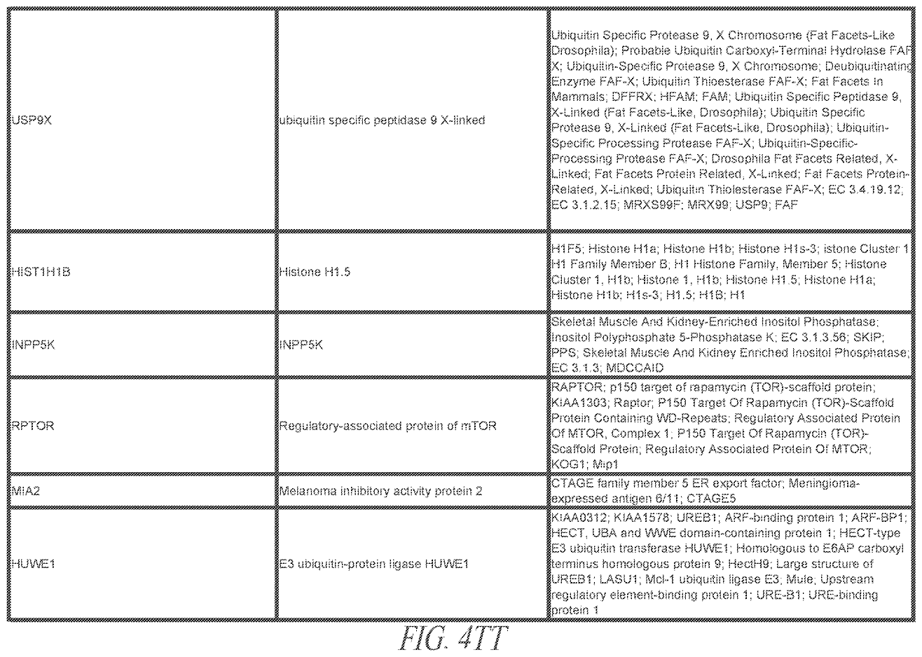

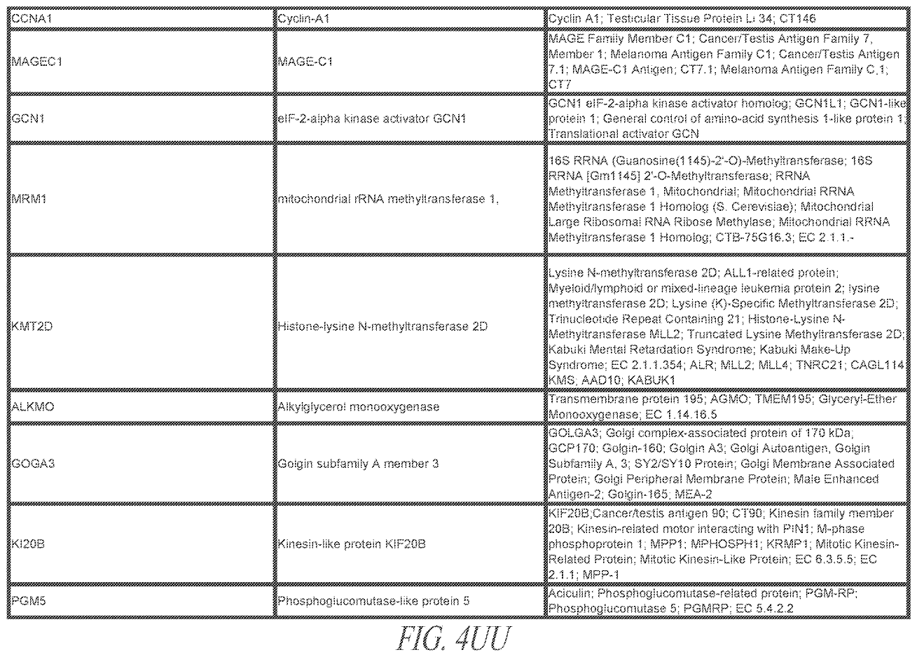

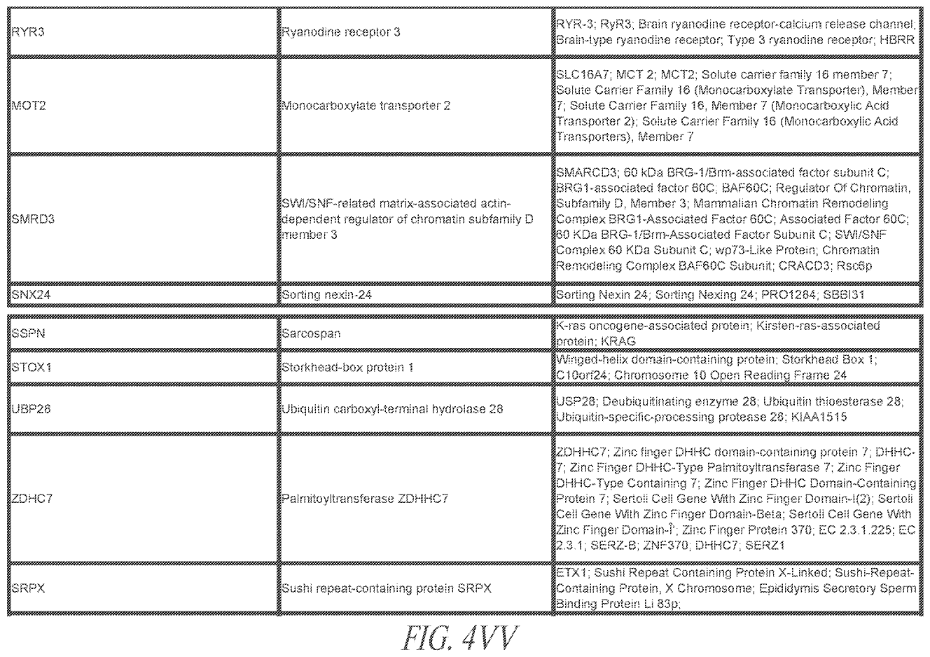

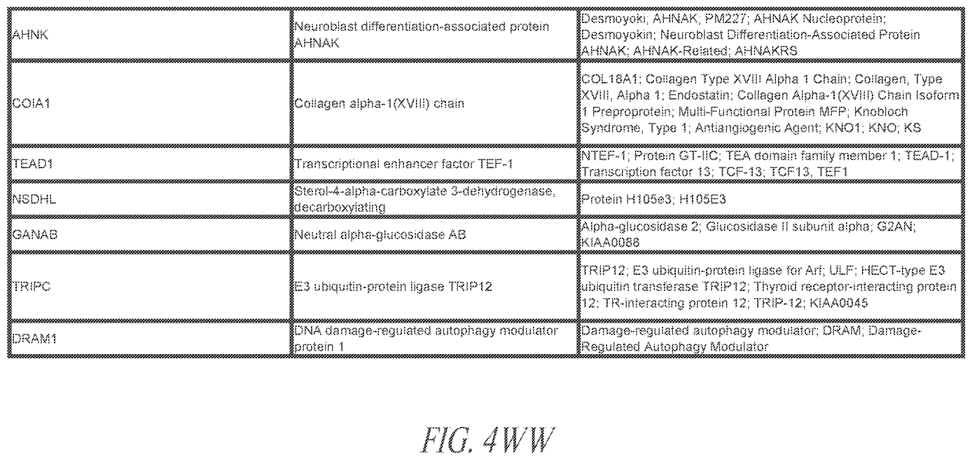

“Tumor antigen” or “tumor antigens” as utilized herein refers to antigens that are presented by MHC class I or class II molecules on the surface of tumor cells. Antigens which are found only on tumor cells are referred to as “Tumor Specific Antigens” or “TSAs,” while antigens that are presented by both tumor cells and normal cells are referred to as “Tumor Associated Antigens” or “TAAs.” Representative examples of tumor antigens include, but are not limited to AIM-2, AIM-3, ART1, ART4, B7-H3, B7-H6, BAGE, β1,6-N, β-catenin, B-cyclin, BMI1, BRAF, BRAP, C13orf24, C6orf153, C9orf112, CA-125, CABYR, CASP-8, cathepsin B, Cav-1, CD70, CD74, CDK-1, CEA, CEAmidkin, COX-2, CRISP3, CSAG2, CSPG4, CTAG2, CYNL2, DHFR, E-cadherin, EGFRvIII, EpCAM, EphA2/Eck, ESO-1, EZH2, FAP, FRα, Fra-1/Fosl 1, FTHL17, GAGE1, Ganglioside/GD2, GD3, GLEA2, GliI, GLUT1, GnT-V, GOLGA, gp75, gplOO, HER-2, HLA-A1+MAGE1, HSPH1, IL-11Ra, IL13Ralpha, IL13Ralpha2, ING4, Ki67, KIAA0376, Ku70/80, LDHC, Lewis-Y, UCAM, Livin, MAGE-A1, MAGE-2, MAGE-A3, MAGE-B6, MAPPK1, MART-1, Mesothelin, MICA, MRP-3, MUC-1, Muc16, MUM-1, Nestin, NKG2D, NKTR, NLRP4, NSEP1, NY-ES-01, OLIG2, p53, PAP, PBK, PRAME, PROX1, PS4, PSCA, PSMA, ras, RBPSUH, ROR1, RTN4, SART1, SART2, SART3, SOX10, SOX11, SOX2, SPANXA1, SSX2, SSX4, SSX5, Survivin, TAG72, TfR1, TNKS2, TPR, TRP-1, TRP-2, TSGA10, TSSK6, TULP2, Tyrosinase, U2AF1L, UPAR, VEGFR2, WT-1, XAGE2, and ZNF165. A more detailed list of tumor antigens is provided in FIGS. 4A to 4WW which provides the antigen name, the common name, as well as synonyms.

Within certain embodiments of the invention, glucose transporter 1 (GLUT1) or transferrin receptor 1 (TfR1) are utilized as cell surface tumor-associated antigens that are broadly expressed in multiple solid tumor types (e.g., the second tumor-associated antigen of the multivalent adapter protein). GLUT1 is overexpressed in multiple solid and hematological malignancies due to its role in facilitating glucose uptake, since increased energy demand is a key hallmark of cancer cells (Adekola K, Rosen S T, Shanmugam M. Glucose transporters in cancer metabolism. Curr Opin Oncol. 2012 November; 24(6):650-4. doi: 10.1097/CCO.0b013e328356da72. PMID: 22913968; PMCID: PMC6392426). Transferrin receptor 1 (TfR1), also known as cluster of differentiation 71 (CD71), is a type II transmembrane glycoprotein that binds transferrin (Tf) and is widely overexpressed in cancers to meet the increased iron requirements of tumor cell proliferation (Shen Y, Li X, Dong D, Zhang B, Xue Y, Shang P. Transferrin receptor 1 in cancer: a new sight for cancer therapy. Am J Cancer Res. 2018 Jun. 1; 8(6):916-931. PMID: 30034931; PMCID: PMC6048407).

An “ectodomain” is the domain of a membrane protein that extends into the extracellular space (i.e. the space outside the target cell). Ectodomains are usually the parts of proteins that initiate contact with surfaces, which leads to signal transduction.

By “specifically binds,” it is generally meant that an antibody or fragment thereof (e.g., an scFv fragment) binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

In order to further an understanding of the various embodiments herein, the following sections are provided which describe various embodiments: A. Oncolytic Viruses; B. Multivalent Engage Proteins; C. Therapeutic Compositions, and D. Administration.

### A. Oncolytic Viruses

As noted above, the present invention provides oncolytic viruses which may be engineered to express and secrete a multivalent adapter protein. Briefly, an “oncolytic virus” is a virus that is capable of replicating in and killing tumor cells. Within certain embodiments the virus can be engineered in order to more selectively target tumor cells. Representative examples of oncolytic viruses include without limitation, adenovirus, coxsackievirus, H-1 parvovirus, herpesviruses such as herpes simplex virus (HSV), influenza virus, measles virus, Myxoma virus, Newcastle disease virus, parvovirus picornavirus, reovirus, rhabdovirus (e.g. vesicular stomatitis virus (VSV)), paramyxovirus such as Newcastle disease virus, picomavirus such as poliovirus or Seneca valley virus, pox viruses such as vaccinia virus (e.g. Copenhagen, Indiana Western Reserve, and Wyeth strains), reovirus, or retrovirus such as murine leukemia virus. Further representative examples are described in: U.S. Pat. Nos. 8,147,822 and 9,045,729 (oncolytic rhabdovirus/VSV); U.S. Pat. No. 9,272,008 (oncolytic Measles virus); U.S. Pat. Nos. 7,223,593, 7,537,924, 7,063,835, 7,063,851, 7,118,755, 8,216,564, 8,277,818, and 8,680,068 (oncolytic herpes virus vectors); and U.S. Pat. No. 8,980,246 (oncolytic vaccinia virus), all of which are incorporated by reference in their entirety.

Within certain embodiments of the invention, the oncolytic virus is Herpes Simplex virus (e.g., HSV-1 or HSV-2). Briefly, Herpes Simplex Virus (HSV) 1 and 2 are members of the Herpesviridae family, which infects humans. The HSV genome contains two unique regions, which are designated unique long (UL) and unique short (Us) region. Each of these regions is flanked by a pair of inverted repeat sequences. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic. Abrogation of neurovirulence may also be achieved by translational control of ICP34.5 using microRNA binding sites inserted into the 3′-untranslated region of ICP34.5 without inactivating both copies of the ICP34.5 gene, or after inactivating only one copy of the ICP34.5 gene and leaving one copy of ICP34.5 to be regulated via microRNA. Neuropathic activity may also be inhibited by mutating the UL37 gene of HSV. Tumor selective replication of HSV may also be conferred by controlling expression of key viral genes such as ICP27 and/or ICP4.

The term “oncolytic herpes virus” or “oHV” refers generally to a herpes virus capable of replicating in and killing tumor cells. The term “oncolytic herpes simplex virus” or “oHSV” refers to a herpes simplex virus that is capable of replicating in and killing tumor cells.

Suitable oncolytic HSV may be derived from either HSV-1 or HSV-2, including any laboratory strain or clinical isolate. In some embodiments, the oHSV may be derived from one of laboratory strains HSV-1 strain 17, HSV-1 strain F, or HSV-2 strain HG52. In other embodiments, it may be derived from non-laboratory strain JS-1. Other suitable HSV-1 viruses include HrrR3 (Goldstein and Weller, J. Viral. 62, 196-205, 1988), G207 (Mineta et al. Nature Medicine. 1(9):938-943, 1995; Kooby et al. The FASEB Journal, 13(11):1325-1334, 1999); G47Delta (Todo et al. Proceedings of the National Academy of Sciences. 2001; 98(11):6396-6401); HSV 1716 (Mace et al. Head & Neck, 2008; 30(8):1045-1051; Harrow et al. Gene Therapy. 2004; 11(22):1648-1658); HF10 (Nakao et al. Cancer Gene Therapy. 2011; 18(3):167-175); NV1020 (Fong et al. Molecular Therapy, 2009; 17(2):389-394); T-VEC (Andtbacka et al. Journal of Clinical Oncology, 2015: 33(25):2780-8); J100 (Gaston et al. PloS one, 2013; 8(11):e81768); M002 (Parker et al. Proceedings of the National Academy of Sciences, 2000; 97(5):2208-2213); NV1042 (Passer et al. Cancer Gene Therapy. 2013; 20(1):17-24); G207-IL2 (Carew et al. Molecular Therapy, 2001; 4(3):250-256); rQNestin34.5 (Kambara et al. Cancer Research, 2005; 65(7):2832-2839); G47A-mIL-18 (Fukuhara et al. Cancer Research, 2005; 65(23):10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

Other representative examples of oncolytic herpes viruses are described in U.S. Pat. Nos. 7,223,593, 7,537,924, 7,063,835, 7,063,851, 7,118,755, 8,216,564, 8,277,818, and 8,680,068, and PCT App. US2022/021798, all of which are incorporated by reference in their entirety.

The oHSV vector has at least one γ34.5 gene that is modified with miRNA target sequences in its 3′ UTR as disclosed herein; there are no unmodified γ34.5 genes in the vector. In some embodiments, the oHSV has two modified γ34.5 genes; in other embodiments, the oHSV has only one γ34.5 gene, and it is modified. In some embodiments, the modified γ34.5 gene(s) are constructed in vitro and inserted into the oHSV vector as replacements for the viral gene(s). When the modified γ34.5 gene is a replacement of only one γ34.5 gene, the other γ34.5 is deleted. Either native γ34.5 gene can be deleted. Alternatively, both native γ34.5 genes can be deleted simultaneously. In one embodiment, the terminal repeat region, which comprises γ34.5 gene and ICP4 gene, is deleted. In another embodiment, the internal repeat region which is identical to the terminal repeat region in wild-type HSV and comprises a second identical copy of the γ34.5 gene and ICP4 gene, is deleted instead of the terminal repeat region. As discussed herein, the modified γ34.5 gene may comprise additional changes, such as having an exogenous promoter.

The oHSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP24, ICP56, ICP8, ICP22, UL5, UL8, UL9, UL30, UL37, UL39/40, UL42. Preferably, a mutation in one of these genes (optionally in both copies of the gene where appropriate) leads to an inability (or reduction of the ability) of the HSV to express the corresponding functional polypeptide. Alternatively, a mutation in one of these genes (optionally in both copies of the gene where appropriate) leads to a change in the functionality of the corresponding polypeptide encoded by said gene or genes. In some embodiments, the promoter of a viral gene may be substituted with a promoter that is selectively active in target cells or inducible upon delivery of an inducer or inducible upon a cellular event or particular environment.

In certain embodiments the expression of ICP4 or ICP27 is controlled by an exogenous promoter, e.g., a tumor-specific promoter. Exemplary tumor-specific promoters include survivin, CEA, CXCR4, PSA, ARR2PB, or telomerase; other suitable tumor-specific promoters may be specific to a single tumor type and are known in the art. Other elements may be present. In some cases, an enhancer such as NFkB/oct4/sox2 enhancer is present. As well, the 5′UTR may be exogenous, such as a 5′UTR from growth factor genes such as FGF.

The oHSV may also have genes and nucleotide sequences that are non-HSV in origin. For example, a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary sequences encode IL12, IL15, IL1S receptor alpha subunit, IL18, OX40L, PD-L1 blocker or a PD-1 blocker. For sequences that encode a product, they are operatively linked to a promoter sequence (e.g., C(CR4 or CMV) and other regulatory sequences (e.g., enhancer, polyadenylation signal sequence) necessary or desirable for expression.

The regulatory region of viral genes may be modified to comprise response elements that affect expression. Exemplary response elements include response elements for NF-κB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

Within certain embodiments of the invention the oncolytic Herpes Virus is as described in PCT/US2022/021798, which is incorporated by reference in its entirety.

### B. Multivalent Adapter Proteins (Also Referred to as “Multivalent Engager Proteins”)

As noted above, the oncolytic viruses of the present invention can be engineered to express and secrete a multivalent adapter protein. In certain embodiments, the multivalent adapter protein includes a first domain that includes a protein or protein fragment that is recognized by an engineered immune cell of interest that comprises a chimeric antigen receptor (e.g., CAR T-cell) or a modified T cell receptor (TCR T-cell), an antibody-drug conjugate (ADC), or anything that utilizes an antibody to target a cell surface target. In other embodiments, the multivalent adapter protein includes a first domain that includes a protein or protein fragment of non-human origin that is recognized by an ADC or an engineered immune cell of interest that comprises a chimeric antigen receptor (e.g., CAR T-cell) or a modified T cell receptor (TCR T-cell). The protein or protein fragment is typically the epitope of the tumor associated antigen that the CAR T-cell, TCR T-cell, or ADC is engineered to specifically bind to (i.e., the first tumor-associated antigen). Because tumor-associated antigens are not 100% restricted to tumor tissue, off-target toxicity may arise due to antigen recognition by the ADC or the engineered immune cell of interest. To avoid potential toxicity resulting from inadvertent activation of the ADC or the engineered immune cell of interest, the chimeric antigen receptor or modified immune cell receptor on the engineered immune cell of interest may be altered to recognize a protein or protein fragment of non-human origin. The protein or protein fragment of non-human origin may be of non-human vertebrate origin, of invertebrate origin, of fungal origin, of arthropod origin, of mollusk origin, of cnidarian origin, of poriferan origin, of archaean origin, of bacterial origin, of protist origin, of plant origin (e. g., gluten), or of viral origin (e. g., a viral surface protein or a fragment thereof). In one embodiment, the protein or protein fragment of non-human origin is the ectodomain of a viral surface protein. In another embodiment, the protein or protein fragment of non-human origin is the ectodomain of a herpes simplex virus (HSV) surface protein, wherein the HSV is either HSV-1 or HSV-2. In yet another embodiment, the protein or protein fragment of non-human origin is the ectodomain of HSV glycoprotein D (gD). In a preferred embodiment, the protein or protein fragment of non-human origin is a synthetic protein or synthetic protein fragment designed in silica to avoid excessive similarity to human proteins in order to reduce the risk of cross-reactivity with human proteins when recognized by an ADC or an engineered immune cell of interest that binds said synthetic protein or synthetic protein fragment. Representative examples of Antibody-Drug Conjugates and methods of making the same are described in, for example: Hamilton G S (September 2015). “Antibody-drug conjugates for cancer therapy: The technological and regulatory challenges of developing drug-biologic hybrids”. Biologicals. 43 (5): 318-32; “Antibody-Drug Conjugates: Methods and Protocols (Methods in Molecular Biology”, 2078) 1st ed. 2020 Edition, Nathan Tumey Ed., Humana Press, ISBN-10 1493999281, ISBN-13 978-1493999286; and “Antibody-Drug Conjugates: The 21st Century Magic Bullets for Cancer” (AAPS Advances in the Pharmaceutical Sciences Series, 17) by Jeffrey Wang (Editor), Wei-Chiang Shen (Editor), Jennica L. Zaro (Editor), ISBN-10 3319376691, ISBN-13 978-3319376691; all of which are incorporated by reference in their entirety.

The engineered immune cell may be a T cell, a NK cell, a macrophage cell, a neutrophil cell, or a B cell.

The multivalent adapter protein also includes a second domain that targets (i.e., specifically binds to) a widely expressed tumor-associated cell surface antigen that is, optionally, ubiquitous across multiple tumor types and is critical for tumor function (i.e., the second tumor-associated antigen). The tumor associated antigen may also be expressed at lower levels by the surrounding non-malignant cells that make up the tumor stroma and other tumor-associated structural components. The multivalent adapter protein may also include a linker moiety that joins the first domain to the second domain. The linker may be any suitable linker structure known in the art. In certain embodiments, the multivalent adapter protein also includes a signal peptide that enables the multivalent adapter protein to be secreted by the host cell infected by the oncolytic virus. The signal peptide may include any suitable signal peptide (i.e., secretory signal) known in the art.

Within one embodiment of the invention the multivalent adapter protein targets a more broadly expressed tumor associated antigen wherein the multivalent adapter/antigen complex remains on the surface of a cell or recycles to the surface of a cell as opposed to being internalized and degraded. Within another embodiment of the invention the multivalent adapter protein targets a more broadly expressed tumor associated antigen wherein the multivalent adapter/antigen complex is internalized within the cell. Suitable second domains of a multivalent adapter protein that target tumor-associated cell surface antigens may be recycling antibodies or antibody fragments such as H7, or internalizing antibodies or antibody fragments such as F12. Within preferred embodiments of the invention the multivalent adapter protein has a second binding domain that recycles to the surface of a cell (e.g., as measured by the assay in Example 5) if >50%, >55%, >60%, >65%, >70%, >75% of antibody is still detectable on the cell surface after 24 hours.

Within other embodiments, the adapter protein is internalized after it is recognized and bound by an ADC in order to maximize ADC efficacy (because ADCs are generally only functional when internalized).

In an exemplary, non-limiting, embodiment, the multivalent adapter protein may have the following amino acid sequence:

(SEQ ID NO: 1)
MEFGLSWVFLVAILKGVQPEEPLVVKVEEGDNAVLQCLKGTSDGPTQQL
TWSRESPLKPFLKLSLGLPGLGIHMRPLAIWLFIFNVSQQMGGFYLCQP
GPPSEKAWQPGWTVNVEGSGELFRWNVSDLGGLGCGLKNRSSEGPSSPS
GKLMSPKLYVWAKDRPEIWEGEPPCLPPRDSLNQSLSQDLTMAPGSTLW
LSCGVPPDSVSRGPLSWTHVHPKGPKSLLSLELKDDRPARDMWVMETGL
LLPRATAQDAGKYYCHRGNLTMSFHLEITARPVLWHWLLRTGGWKGGGG
SGGGGSGGGGSQVQLQESGGGVVQPGRSLRLSCAASRFTFSSYAMHWVR
QAPGKGLEWVAVISYDGSNKYYADSVKGRFTISRDNSKNTLYLQMNSLR
AEDTAVYYCARDLSGYGDYPDYWGQGTLVTVSSGGGGSGGGGSGGGGSS
ELTQDPAVSVALGQTVRITCQGDSLRSYYASWYQQKPGQAPVLVMYGRN
ERPSGVPDRFSGSKSGTSASLAISGLQPEDEANYYCAGWDDSLTGPVFG
GGTKLTVL

The multivalent adapter protein of SEQ ID NO:1 includes the following elements:
Signal peptide:

(SEQ ID NO: 2)
MEFGLSWVFLVAILKGVQ.

Ectodomain:

(SEQ ID NO: 3)
PEEPLVVKVEEGDNAVLQCLKGTSDGPTQQLTWSRESPLKPFLKLSLGL
PGLGIHMRPLAIWLFIFNVSQQMGGFYLCQPGPPSEKAWQPGWTVNVEG
SGELFRWNVSDLGGLGCGLKNRSSEGPSSPSGKLMSPKLYVWAKDRPEI
WEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRG
NLTMSFHLEITARPVLWHWLLRTGGWK.

Linker:

(SEQ ID NO: 4)
GGGGSGGGGSGGGGS.

Tumor-Associated Antigen Binding Domain:

(SEQ ID NO: 5)
QVQLQESGGGVVQPGRSLRLSCAASRFTFSSYAMHWVRQAPGKGLEWVA
VISYDGSNKYYADSVKGRFTISRDNSKNTLYLQMNSLRAEDTAVYYCAR
DLSGYGDYPDYWGQGTLVTVSSGGGGSGGGGSGGGGSSELTQDPAVSVA
LGQTVRITCQGDSLRSYYASWYQQKPGQAPVLVMYGRNERPSGVPDRFS
GSKSGTSASLAISGLQPEDEANYYCAGWDDSLTGPVFGGGTKLTVL.

In another embodiment of the invention a multivalent engager molecule termed “gD-H7” is also provided:

(SEQ ID NO: 6)
MEFGLSWVFLVAILKGVQCGGLINKYALVDASLKMADPNRFRGKDLPVL
DQLTDPPGVRRVYHIQAGLPDPFQPPSLPITVYYAVLERACRSVLLNAP
SEAPQIVRGASEDVRKQPYNLTIAWFRMGGNCAIPITVMEYTECSYNKS
LGACPIRTQPRWNYYDSFSAVSEDNLGFLMHAPAFETAGTYLRLVKIND
WTEITQFILEHRAKGSCKYALPLRIPPSACLSPQAYQQGVTVDSIGMLP
RFIPENQRTVAVYSLKIAGWHGPKAPYTSTLLPPELSETPNATQPELAP
EDPEDSALLEDPVGTVAPQIPPNWHIPSIQDAATPYGAPGGGGSGGGGS
GGGGSQVQLQESGGGVVQPGRSLRLSCAASRFTFSSYAMHWVRQAPGKG
LEWVAVISYDGSNKYYADSVKGRFTISRDNSKNTLYLQMNSLRAEDTAV
YYCARDLSGYGDYPDYWGQGTLVTVSSGGGGSGGGGSGGGGSSELTQDP
AVSVALGQTVRITCQGDSLRSYYASWYQQKPGQAPVLVMYGRNERPSGV
PDRFSGSKSGTSASLAISGLQPEDEANYYCAGWDDSLTGPVFGGGTKLT
VLG.

The multivalent adapter protein of SEQ ID NO:6 includes the following elements:

(SEQ ID NO: 2)
MEFGLSWVFLVAILKGVQ.

gD ectodomain is:

(SEQ ID NO: 7)
KYALVDASLKMADPNRFRGKDLPVLDQLTDPPGVRRVYHIQAGLPDPFQ
PPSLPITVYYAVLERACRSVLLNAPSEAPQIVRGASEDVRKQPYNLTIA
WFRMGGNCAIPITVMEYTECSYNKSLGACPIRTQPRWNYYDSFSAVSED
NLGFLMHAPAFETAGTYLRLVKINDWTEITQFILEHRAKGSCKYALPLR
IPPSACLSPQAYQQGVTVDSIGMLPRFIPENQRTVAVYSLKIAGWHGPK
APYTSTLLPPELSETPNATQPELAPEDPEDSALLEDPVGTVAPQIPPNW
HIPSIQDAATPY.

GS linker is:

(SEQ ID NO: 4)
GGGGSGGGGSGGGGS.

H7 scFv designed to target TfR1 is:

(SEQ ID NO: 8)
QLQESGGGVVQPGRSLRLSCAASRFTFSSYAMHWVRQAPGKGLEWVAVI
SYDGSNKYYADSVKGRFTISRDNSKNTLYLQMNSLRAEDTAVYYCARDL
SGYGDYPDYWGQGTLVTVSSGGGGSGGGGSGGGGSSELTQDPAVSVALG
QTVRITCQGDSLRSYYASWYQQKPGQAPVLVMYGRNERPSGVPDRFSGS
KSGTSASLAISGLQPEDEANYYCAGWDDSLTGPVFGGGTKLTVLG.

In another exemplary, non-limiting, embodiment, a multivalent adapter molecule termed “HER2-F12” is also provided:

MEFGLSWVFLVAILKGVQTQVCTGTDMKLRLPASPETHLDMLRHLYQGC
QVVQGNLELTYLPTNASLSFLQDIQEVQGYVLIAHNQVRQVPLQRLRIV
RGTQLFEDNYALAVLDNGDPLNNTTPVTGASPGGLRELQLRSLTEILKG
GVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKH
SDCLACLHFNHSGICELHCPALVTYNTDTFESMPNPEGRYTFGASCVTA
CPYNYLSTDVGSCTLVCPLHNQEVTAEDGTQRCEKCSKPCARVCYGLGM
EHLREVRAVTSANIQEFAGCKKIFGSLAFLPESFDGDPASNTAPLQPEQ
LQVFETLEEITGYLYISAWPDSLPDLSVFQNLQVIRGRILHNGAYSLTL
QGLGISWLGLRSLRELGSGLALIHHNTHLCFVHTVPWDQLFRNPHQALL
HTANRPEDECVGEGLACHQLCARGHCWGPGPTQCVNCSQFLRGQECVEE
CRVLQGLPREYVNARHCLPCHPECQPQNGSVTCFGPEADQCVACAHYKD
PPFCVARCPSGVKPDLSYMPIWKFPDEEGACQPCPINCTHSCVDLDDKG
CPAEQRASPLTAPGGGGSGGGGSGGGGSQVQLQESGGGLVQPGGSLRLS
CAASGFSFNTYTMHWVRQAPGKGLEWVADIAYDGSTKYYADSVKGRFTI
SRDNAKNSLYLQMNSLRAEDTAVYYCARDAVAGEGYFDLWGRGTLVTVS
SGGGGSGGGGSGGGGSQSALTQDPAVSVALGQTVRITCQGDSLRSYYAS
WYQQLPGTAPKLLIYRNNQRPSGVPDRFSGSKSGTSASLAISGLRSEDE
ADYYCAAWDDSLSAWVFGGGTKLTVL.

The multivalent adapter molecule “HER2-F12” Includes the following elements:
Signal peptide is:

MEFGLSWVFLVAILKGVQ

TQVCTGTDMKLRLPASPETHLDMLRHLYQGCQVVQGNLELTYLPTNASL
SFLQDIQEVQGYVLIAHNQVRQVPLQRLRIVRGTQLFEDNYALAVLDNG
DPLNNTTPVTGASPGGLRELQLRSLTEILKGGVLIQRNPQLCYQDTILW
KDIFHKNNQLALTLIDTNRSRACHPCSPMCKGSRCWGESSEDCQSLTRT
VCAGGCARCKGPLPTDCCHEQCAAGCTGPKHSDCLACLHFNHSGICELH
CPALVTYNTDTFESMPNPEGRYTFGASCVTACPYNYLSTDVGSCTLVCP
LHNQEVTAEDGTQRCEKCSKPCARVCYGLGMEHLREVRAVTSANIQEFA
GCKKIFGSLAFLPESFDGDPASNTAPLQPEQLQVFETLEEITGYLYISA
WPDSLPDLSVFQNLQVIRGRILHNGAYSLTLQGLGISWLGLRSLRELGS
GLALIHHNTHLCFVHTVPWDQLFRNPHQALLHTANRPEDECVGEGLACH
QLCARGHCWGPGPTQCVNCSQFLRGQECVEECRVLQGLPREYVNARHCL
PCHPECQPQNGSVTCFGPEADQCVACAHYKDPPFCVARCPSGVKPDLSY
MPIWKFPDEEGACQPCPINCTHSCVDLDDKGCPAEQRASPLTAP.

HER2 ectodomain is:
GS linker is:

(SEQ ID NO: 4)
GGGGSGGGGSGGGGS.

F12 scFv designed to target TfR1 is:

QVQLQESGGGLVQPGGSLRLSCAASGFSFNTYTMHWVRQAPGKGLEWVA
DIAYDGSTKYYADSVKGRFTISRDNAKNSLYLQMNSLRAEDTAVYYCAR
DAVAGEGYFDLWGRGTLVTVSSGGGGSGGGGSGGGGSQSALTQDPAVSV
ALGQTVRITCQGDSLRSYYASWYQQLPGTAPKLLIYRNNQRPSGVPDRF
SGSKSGTSASLAISGLRSEDEADYYCAAWDDSLSAWVFGGGTKLTVL.

All other regions in SEQ ID NO:6 are cloning sites that are not relevant to the function of the multivalent engager molecule.

Within another embodiment of the invention, the multivalent adapter molecules may include a GS linker, a GGS linker, a GGGS linker, a GGGGS linker, a GGGGSGGGGS linker, a GGGGSGGGGSGGGGS linker, a signal peptide MEFGLSWVFLVAILKGVK, a signal peptide MEFGLSWVFLVAILKGVQ, or a signal peptide MEFGLSWVFLVAILKGVQC.

In certain embodiments, the broadly expressed tumor associated antigen (i.e., the second tumor-associated antigen) may be TfR1. The use of TfR1 as a traditional CAR T-cell therapy target is problematic because it is also expressed on healthy cells, albeit at a lower level. A further complication is that TfR1 expression is upregulated on activated T-cells, resulting in the mutual destruction of TfR-CAR T-cells (Guo Z, Zhang Y, Fu M, Zhao L, Wang Z, Xu Z, Zhu H, Lan X, Shen G, He Y, Lei P. The Transferrin Receptor-Directed CAR for the Therapy of Hematologic Malignancies. Front Immunol. 2021 Mar. 29; 12:652924. doi: 10.3389/fimmu.2021.652924. PMID: 33854512; PMCID: PMC8039461). One advantage of the present invention avoids these pitfalls by introducing the oncolytic virus expressing the TfR1-targeting multivalent engager prior to CAR T-cell infusion, which allows the engager to spread locally within the tumor microenvironment and coat the heterogeneous tumor cell mixture and tumor stroma with TAA ectodomains that will be targeted for destruction via the TAA-specific CAR.

Because cells infected with oncolytic virus are already destined for destruction, in some embodiments short hairpin RNA (shRNA) mediated gene silencing may be utilized to reduce or eliminate expression of the broadly expressed tumor associated antigen (i.e., the second tumor-associated antigen) such as TfR1 from infected cells in order to extend the length of productive viral infection by protecting infected cells from premature death via retargeted immune cells. To that end, shRNA targeting the second tumor-associated antigen (e.g., TfR1 or GLUT1) may also be expressed by the same oncolytic virus that is engineered to express the multivalent adapter protein.

As an alternative to using an oncolytic virus to express and secrete the multivalent adapter protein, in some embodiments the multivalent adapter protein may be encoded within an mRNA molecule that is encapsulated within lipid nanoparticles or other forms of delivery systems and injected into the tumor for internalization and translation. Tumor-specific expression of the multivalent adapter protein from said mRNA may be achieved by adding miRNA target sequences to the 3′-end and/or the 5′-end of said mRNA, wherein said miRNA target sequences are recognized by miRNAs that are less abundant in the targeted tumor cells compared to normal cells. The adapter may also be delivered intratumorally either by direct injection of the purified adapter protein or by injecting RNA encoding the adapter (as LNP-encapsulated mRNA, self-amplifying RNA, etc.).

Antibody binding to TfR1 normally triggers internalization and eventual destruction of the antibody. To overcome this problem, in certain embodiments, the multivalent adapter proteins of the present invention may include all or a portion of the recycling anti-TfR1 monoclonal antibody H7 (refer to Neiveyans M, Melhem R, Arnoult C, Bourquard T, Jarlier M, Busson M, Laroche A, Cerutti M, Pugnière M, Temant D, Gaborit N, Chardès T, Poupon A, Gouilleux-Gruart V, Pèlegrin A, Poul M A. A recycling anti-transferrin receptor-1 monoclonal antibody as an efficient therapy for erythroleukemia through target up-regulation and antibody-dependent cytotoxic effector functions. MAbs. 2019 April; 11(3):593-605. doi: 10.1080/19420862.2018.1564510. Epub 2019 Feb. 18. PMID: 30604643; PMCID: PMC6512944, which is incorporated by reference in its entirety), which can dissociate from its bound antigen in the acidic environment of the sorting endosomes and avoid lysosomal degradation, while recycling back to be displayed on the plasma membrane.

For retargeting ADCs, a non-recycling antibody that can internalize to enable intracellular delivery of the ADC may be preferable. To overcome this problem, in certain embodiments, the multivalent adapter proteins of the present invention may include all or a portion of the non-recycling anti-TfR1 monoclonal antibody F12 (refer to Neiveyans M, Melhem R, Arnoult C, Bourquard T, Jarlier M, Busson M, Laroche A, Cerutti M, Pugnière M, Temant D, Gaborit N, Chardès T, Poupon A, Gouilleux-Gruart V, Pèlegrin A, Poul M A. A recycling anti-transferrin receptor-1 monoclonal antibody as an efficient therapy for erythroleukemia through target up-regulation and antibody-dependent cytotoxic effector functions. MAbs. 2019 April; 11(3):593-605. doi: 10.1080/19420862.2018.1564510. Epub 2019 Feb. 18. PMID: 30604643; PMCID: PMC6512944, which is incorporated by reference in its entirety).

### C. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein, as well as compositions comprising an oncolytic virus or a multivalent engager as described herein and CAR T-cells, CAR NK-cells, CAR macrophage cells, CAR neutrophils, or engineered T cell receptors, or antibody drug conjugates that target antigens present on solid tumors (where the antigens have a well-defined ectodomain that can be used for retargeting via the multivalent engager payload as described herein). When combined with these cells or antibody drug conjugates, the multivalent engager may be delivered directly as a purified protein (e.g., via intratumoral injection, delayed release pellet implantation, etc.), the multivalent engager may be encoded self-amplifying RNA or encoded within an mRNA molecule that is further encapsulated within lipid nanoparticles or other forms of delivery systems and directly injected to target tissues for internalization and translation, or the multivalent engager may be delivered by a vehicle. The vehicle may be an expression vector that encodes the multivalent engager. The vehicle may be a viral vector, comprising oncolytic or non-oncolytic viruses, e.g., a retroviral vector, a lentiviral vector, an adenoviral vector, a herpes simplex virus vector, or a chimeric viral vector. Within a preferred embodiment, the multivalent engager is delivered to the tumor by encoding the multivalent engager within HSV (HSV-1 or HSV-2).

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States Pharmacopeia: The National Formulary (USP 40-NF 35 and Supplements).

In the case of the oncolytic viruses and CAR T-cells as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the OV to a cancer cell and/or CAR T-cells will preferably be administered without undue toxicity.

The compositions provided herein can be provided at a variety of concentrations. For example, dosages of oncolytic virus can be provided which range from about 10⁴ pfu to about 10¹⁰ pfu. Within further embodiments, the dosage can range from about 10⁶ pfu to about 10⁷ pfu, or from about 10⁷ pfu to about 10⁸ pfu, or from about 10⁸ pfu to 10⁹ pfu, and may be administered as a single dose or as multiple doses spread out over time. Doses may be administered daily, weekly, biweekly, monthly, or bimonthly, and dosage frequency may be cyclical, with each cycle comprising a repeating dosage pattern (e. g. once a week or biweekly dose administration for about 4 weeks comprising one cycle, repeating for up to about 24 cycles). Within other embodiments of the invention, the virus can be provided in ranges from about 5×10⁴ pfu/kg to about 2×10⁹ pfu/kg for intravenous delivery in humans. For intratumoral injection, the preferred dosage can range from about 10⁶ pfu to about 10⁹ pfu per dose (with an injectable volume which ranges from about 0.1 mL to about 5 mL).

Within certain embodiments of the invention, lower or higher dosages than standard may be utilized. Hence, within certain embodiments less than about 10⁶ pfu or more than about 10⁹ pfu can be administered to a patient.

The CAR T-cells (or CAR NK-cells, or CAR-macrophages, etc.) are prepared and reinfused back into the patient using standard protocols. The present application focuses on retargeting the CAR-modified or TCR-modified immune cells regardless of how they were constructed or administered. The CAR-modified or TCR-modified immune cells may be administered either intratumorally or intravenously or intraperitoneally or intramuscularly or subcutaneously. By contrast, the oncolytic virus or mRNA or other delivery vehicle carrying the multivalent engager molecule can be administered intratumorally to reduce the risk of off-target effects since the multivalent engager payload is designed to locally expand the target range of the adoptive cell therapy.

The compositions may be stored at a temperature conducive to stable shelf-life and includes room temperature (about 20° C.), 4° C., −20° C., −80° C., and in liquid N2. Because compositions intended for use in vivo generally do not have preservatives, storage will generally be at colder temperatures. Compositions may be stored dry (e.g., lyophilized) or in liquid form.

### D. Administration

In addition to the compositions described herein, various methods of using such compositions to treat or ameliorate cancer are provided, comprising the step of administering an effective dose or amount of oHSV as described herein to a subject.

The terms “effective dose” and “effective amount” refers to amounts of the oncolytic virus and CAR T-cells that are sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemias, lymphomas, myelomas, and sarcomas. Representative forms of leukemias include acute myeloid leukemia (AML) and representative forms of lymphoma include B cell lymphomas. Further examples include, but are not limited to cancer of the bile duct, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemias and lymphomas), kidney, larynx, lung, liver, oral cavity, ovaries, pancreas, prostate, skin (e.g., melanoma and squamous cell carcinoma), GI (e.g., esophagus, stomach, and colon) and thyroid. Cancers can comprise solid tumors (e.g., sarcomas such as fibrosarcoma, myxosarcoma, liposarcoma, chondrosarcoma and osteogenic sarcoma), be diffuse (e.g., leukemias), or some combination of these (e.g., a metastatic cancer having both solid tumors and disseminated or diffuse cancer cells).

Within certain embodiments of the invention the cancer can be resistant to or refractory from conventional treatment (e.g. conventional chemotherapy and/or radiation therapy). Benign tumors and other conditions of unwanted cell proliferation may also be treated.

The recombinant oncolytic viruses described herein may be given by a route that is e.g. oral, topical, parenteral, systemic, intravenous, intramuscular, intraocular, intrathecal, intratumoral, subcutaneous, or transdermal. Within certain embodiments the oncolytic virus may be delivered by a cannula, by a catheter, or by direct injection. The site of administration may be directly into the tumor, adjacent to the tumor, or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as administration of a different oncolytic virus, radiotherapy, administration of a checkpoint inhibitor, chemotherapy using, e.g., a chemotherapeutic agent such as etoposide, ifosfamide, adriamycin, vincristine, doxycycline, and others.

Recombinant oncolytic viruses described herein may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional.

A therapeutically effective amount is preferably administered. This is an amount that is sufficient to show benefit to the subject. The actual amount administered, and the time-course of administration will depend at least in part on the nature of the cancer, the condition of the subject, site of delivery, and other factors.

Within yet other embodiments of the invention the oncolytic virus can be administered by a variety of methods, e.g., intratumorally, or, after surgical resection of a tumor.

The following examples are offered by way of illustration, and not by way of limitation.

### EXAMPLES

### Example 1

Use of an Oncolytic Virus-Delivered Secretable Multivalent Adapter Protein to Tag Cells for Immune Destruction

Objective: The objective of this protocol is to exemplify one method for treating cancer by targeting and destroying cells in a tumor mass, including tumor-associated and stromal cells, even if the cells avoid direct infection by the oncolytic virus (OV) and do not normally express a tumor-associated antigen (TAA) targeted by CAR T cells.

Study design: Traditional challenges in treating solid tumors with CAR-T cell and/or OV therapies include the fact that OV infection only occurs within a subset of tumor cells, and most tumor-associated cells also lack the highly specific tumor antigens used for CAR targeting. As disclosed herein, clinically proven CAR T-cells, designed to target a specific tumor antigen, such as CD19, HER2 or EGFR, can be retargeted in a highly localized fashion to destroy a broad range of cells within the tumor mass that do not normally express CD19, HER2 or EGFR on the cell surface. This is accomplished by intratumoral administration of an oncolytic virus expressing a secretable multivalent engager molecule.

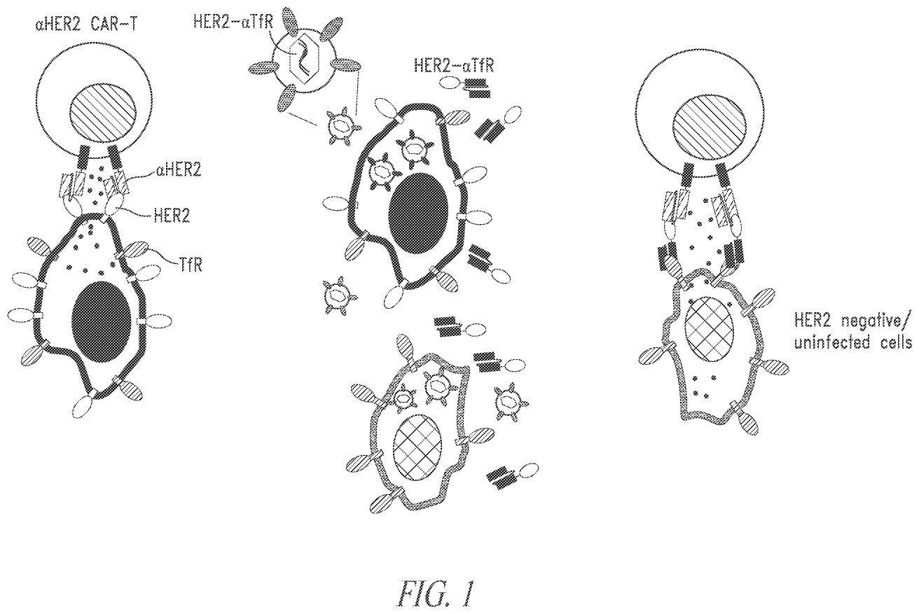

This study outlines the combined use of HER2-CAR adoptive T-cell immunotherapy and an oncolytic virus expressing and secreting a multivalent adapter protein, HER2-αTFR and is summarized by the illustration in FIG. 1. The multivalent adapter protein includes a HER2 ectodomain that is recognized by the HER2-CAR to trigger T cell activation and a TfR1-targeting scFv designed to bind the ubiquitous TfR1 receptor that is overexpressed in nearly all tumor cells. Crucially, the use of a recycling antibody for the anti-TfR1 arm of the multivalent engager ensures that the engager molecule is always displayed on the surface of target cells without being internalized and degraded.

The oncolytic virus can infect many cell types within the tumor, regardless of their level of TAA expression. Even if the number of cells infected is small and if the virus does not persist for long before clearance by the anti-viral immune response, the viral infection will potentiate expression and localized secretion of the multivalent engager payload that will bind to TfR1 receptors on the surface of most tumor and tumor-associated cells even if the cells have not been infected and have no CAR-targeted TAA expressed on their surface.

Cells decorated with the multivalent engager HER2-αTFR can then be recognized and destroyed by HER2-CART-cells and this strategy can be employed for any readily available CAR T-cell therapy by simply modifying the T-cell engager arm of the multivalent engager to match the TAA epitope targeted by the CAR. In other embodiments, the CAR targets a highly tumor-specific epitope, such as HER2, instead of an epitope that is found on both tumor and healthy cells, such as CD19, in order to minimize toxicity due to off-target effects.

Extreme tumor specificity in adoptive T-cell immunotherapy typically has several drawbacks, chief among them being the limited number of tumors that can be targeted and the fact that tumor-associated cells and tumor stroma are unlikely to express the target TAA. In contrast, as described herein, the present invention provides a strategy of using an OV-delivered multivalent engager that transforms these drawbacks into strengths by leveraging highly tumor-specific CARs to minimize off-target toxicity while enabling localized broad-spectrum tumor recognition and destruction in tumors that have been labeled with the TfR1-targeting multivalent engager molecule. Other surface targets such as GLUT1 may also be used for the multivalent engager instead of TfR1, as long as the target is broadly overexpressed in malignant tissues and is therefore risky to use in traditional CAR T-cell therapy due to the likelihood of destroying normal tissues that also express said target antigen.

### Example 2

### Assay for Determining the Ability of Antibody-Coated HSV-1 Virus to Infect Tumor Cells In Vitro

Objective: To test the efficacy of tumor cell killing by CD19-CAR T cells in tumor cells that do not normally express CD19, but which have been decorated on the cell surface by the CD19-H7 multivalent engager molecule binding to the ubiquitous TfR1 tumor-associated antigen.

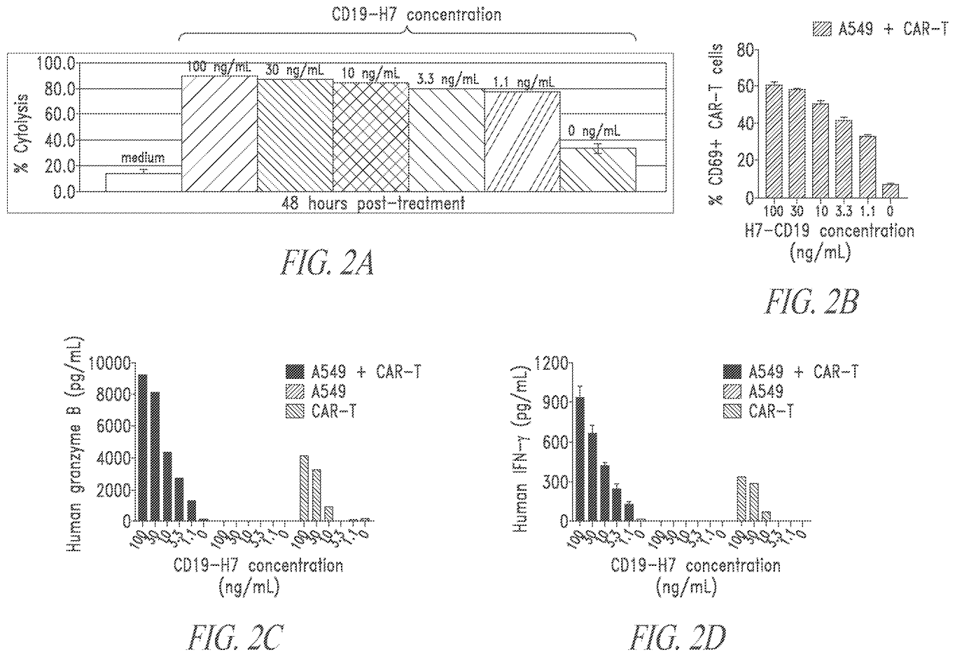

Study design: The multivalent engager molecule, CD19-H7 (SEQ ID NO: 1) was constructed with one engager arm consisting of the CD19 ectodomain (SEQ ID NO:3) and the other engager arm consisting of the H7 scFv designed to target TfR1 (SEQ ID NO:5). A549 cells were seeded and co-incubated with purified CD19-H7 protein in the presence of CAR T-cells engineered to target CD19. Medium alone was used as the negative control. 48 hours later, A549 cell cytotoxicity was monitored in real-time by xCelligence eSight (FIG. 2A) and CD69 upregulation on the CART cell surface was measured by flow cytometry (FIG. 2B). Cell culture supernatants from the same cell-based assay setting were harvested and the production of human granzyme B (FIG. 2C) and human IFN-γ (FIG. 2D) was quantified by ELISA assays.

Results: Robust cytotoxicity of A549 cells was observed after treatment, even with the lowest tested concentration of the CD19-H7 multivalent engager. Treatment with CD19-H7 and co-incubation with CD19-CAR T cells resulted in significant T cell activation when compared to untreated control CD19-CAR T cells, as evidenced by increased CD69 upregulation and enhanced production of human granzyme B and human IFN-γ.

Conclusions: The CD19-H7 multivalent adapter protein can successfully bind to the TfR1 receptor on CD19-negative A549 cells and activate CD19-CAR T-cells, triggering increased cytokine production and A549 cell killing.

### Example 3

### Assay for Measuring Activation of CAR-Jurkat Cells Using Multivalent Engagers on Different Tumor Cells

Objective: Jurkat cells are an immortalized line of human T lymphocyte cells that are commonly used to study T cell signaling. A customized Jurkat cell line engineered with a chimeric antigen receptor targeting HER2 was used to test the in vivo efficacy of a multivalent engager molecule with one arm comprising a HER2 protein fragment and the other arm comprising an antibody targeting the transferrin receptor.

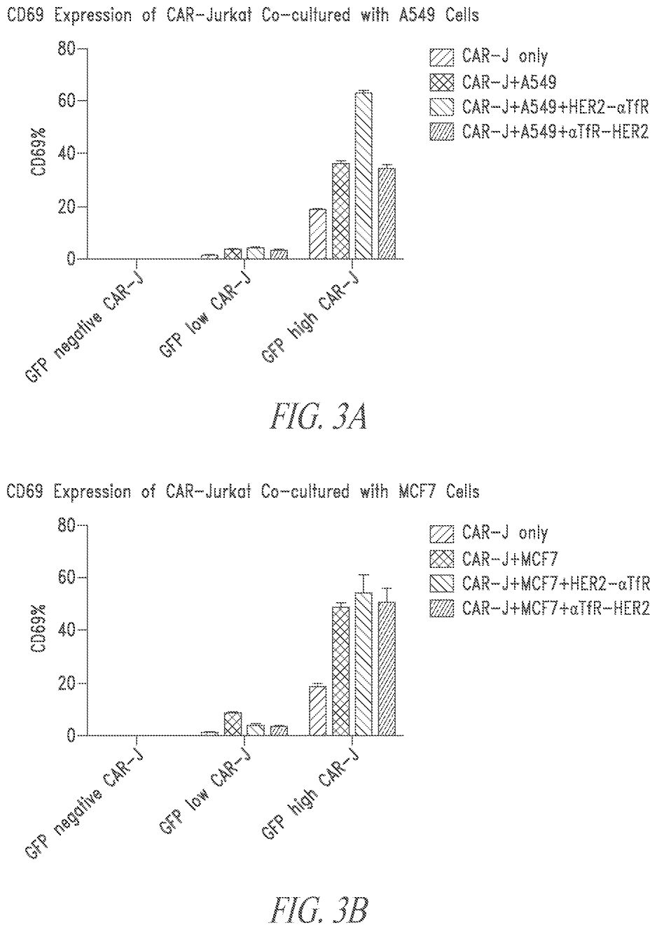

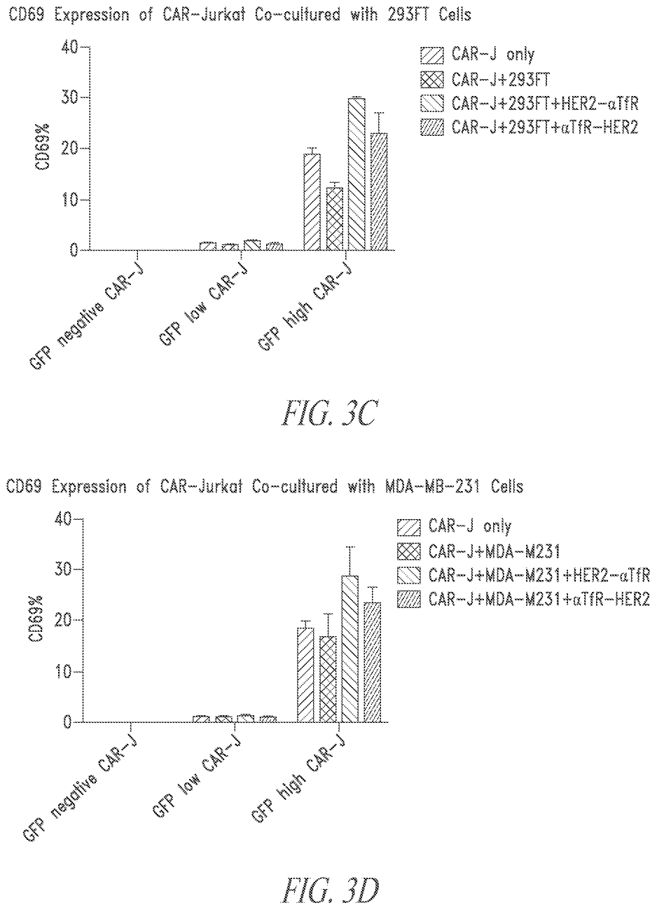

Study design: αHER2-CAR-Jurkat cells were generated with a lentivirus expressing αHER2-CAR-GFP. Three different populations of αHER2-CAR-Jurkat cells were observed based on the expression of GFP. The CAR-Jurkat engagers, HER2-αTfR (anti-transferrin receptor) and αTfR-HER2, were produced from Expi293 cells. CAR-Jurkat cells were incubated with or without the engager and different tumor cell lines including HER2+ A549 (FIG. 3A), HER2+ MCF7 (FIG. 3B), HER2− 293FT (FIG. 3C) and HER2− MDA-MB-231 (FIG. 3D) for 24 hours. Flow cytometry was used to assess the expression of CD69.

Results: As shown in FIG. 3, low levels of GFP expression correlated with minimal CD69 expression, consistent with unsuccessful incorporation of the chimeric antigen receptor in low-GFP αHER2-CAR-Jurkat cells. Co-incubation of high-GFP αHER2-CAR-Jurkat cells with HER2+ cell lines A549 and MCF7 resulted in a marked increase in CD69 expression consistent with T cell activation. Addition of HER2-αTfR multivalent engager yielded another ^{˜}20% Increase in CD69 expression on A549 cells, but no corresponding increase in CD69 expression was observed on MCF7 cells. Use of the αTfR-HER2 multivalent engager did not lead to increases in CD69 expression compared to A549 cells or MCF7 cells alone. By contrast, co-incubation of high-GFP αHER2-CAR-Jurkat cells with HER2-negative cell lines 293FT and MDA-MB-231 did not lead to increased CD69 expression until the multivalent engager was added. Both variants of multivalent engager were effective at triggering CD69 upregulation, although the HER2-αTfR multivalent engager was approximately 10% more effective than αTfR-HER2.

Conclusions: Engineered αHER2-CAR-Jurkat cells exhibited increased activation as measured by CD69 expression in the presence of HER2-positive cells. However, even HER2-negative cells were able to trigger αHER2-CAR-Jurkat cell activation following co-incubation with a secretable multivalent engager comprising a HER2 ectodomain fused to an anti-TfR scFv. Moreover, the orientation of elements within the multivalent engager was found to be significant because the HER2-αTfR multivalent engager was more effective than the αTfR-HER2 multivalent engager variant in all tested scenarios.

### Example 4

### Effect of gD-H7 Decoration on Induction of CAR T-Cell Function in A549 Tumor Cells

Objective: To test the efficacy of tumor cell killing by anti gD-CAR T-cells in tumor cells that do not normally express HSV-1 glycoprotein D (gD) in the absence of HSV infection, but which have been decorated on the cell surface by the gD-H7 multivalent engager molecule binding to the ubiquitous TfR1 tumor-associated antigen.

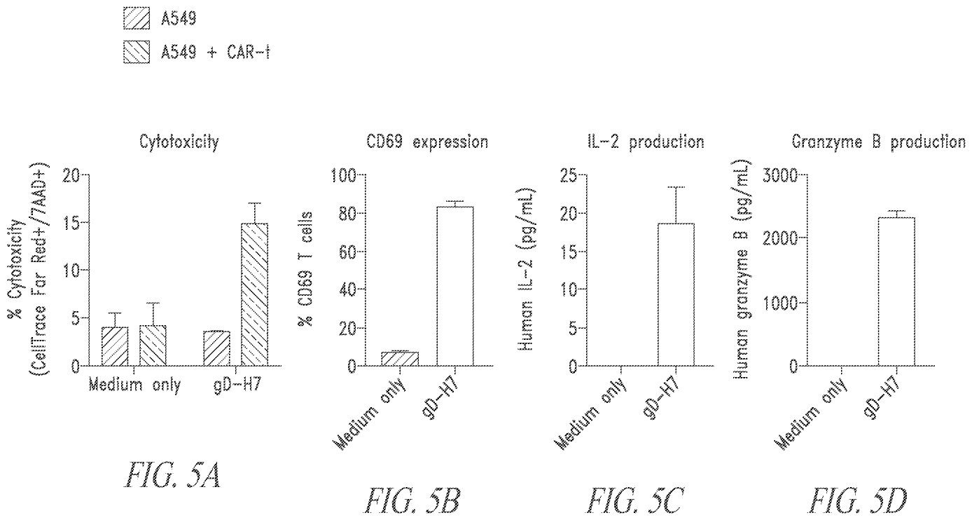

Study design: The multivalent engager molecule, gD-H7 (see SEQ ID NO:6) was constructed with one engager arm consisting of the gD ectodomain and the other engager arm consisting of the H7 scFv designed to target TfR1. A549 tumor cells (1.2×10⁴ cells per well) were labeled with CellTrace Far Red dye and seeded in a 96-well flat-bottom plate at 37° C. overnight, followed by co-incubation with purified gD-H7 protein in the presence of CAR T-cells (1.2×10⁵ cells per well) engineered to target gD, which is normally absent on A549 tumor cells. Medium alone was used as the negative control. 48 hours later, A549 cell cytotoxicity was assessed by 7AAD staining (FIG. 5A), and CAR T-cell activation was measured by quantifying CD69 upregulation on the CAR T-cell surface by flow cytometry (FIG. 5B). Cell culture supernatants from the same cell-based assay setting were harvested and the production of human IL-2 (FIG. 5C), and human granzyme B (FIG. 5D) was quantified by ELISA assays.

Results: Robust cytotoxicity of A549 cells was observed after treatment, even with the lowest tested concentration of the gD-H7 multivalent engager. Treatment with gD-H7 and co-incubation with anti-gD CAR T-cells resulted in significant T cell activation when compared to untreated control anti-gD CAR T-cells, as evidenced by increased CD69 upregulation and enhanced production of human granzyme B and human IL-2.

Conclusions: The gD-H7 multivalent adapter protein can successfully bind to the TfR1 receptor on gD-negative A549 cells and activate anti-gD CAR T-cells, triggering increased cytokine production and A549 cell killing.

### Example 5

### Antibody Recycling Assay to Evaluate Antibodies for Use as the Antigen-Binding Arm of the Multivalent Adapter Molecule

Cells expressing a targeted receptor are incubated on ice with the antibody of interest which targets the receptor. Subsequently, antibody-labeled cells are washed and then transferred and incubated at 37 degrees Celsius for desired time durations (0 hours, 6 hours, 12 hours, 18 hours, 24 hours, 30 hours, 36 hours, etc.). At the end of each time point, the antibody-labeled cells are collected and subjected to flow cytometry to check for presence of the antibody on the cell surface. If >50%, >55%, >60%, >65%, >70%, >75%) of antibody is still detectable on the cell surface after 24 hours, it can be defined as a “recycling antibody”.

### Example 6

### Effect of VG22001 Expressing CD19-αTfR and VG2003 without CD19-αTfR in A549 Cells, when Co-Cultured with CD19-CAR-T Cells or Mock T Cells

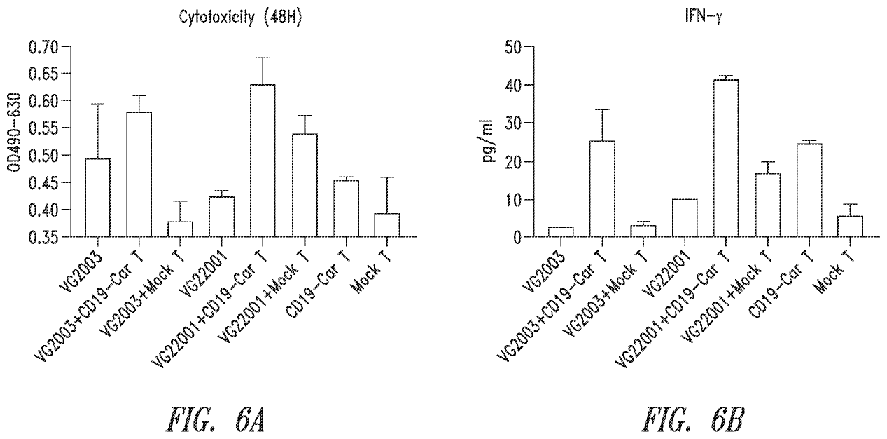

Study design: The viruses VG22001 (expressing CD19-αTfR) and VG2003 (without CD19-αTfR) were co-cultured with the human lung cancer cell line A549 at an MOI=0.1 for 24 hours. CD19-CAR-T cells or Mock T cells were then added into the virus infected A549 cells (E:T=5:1). After 48 hours, lactate dehydrogenase in cell supernatants was detected to determine cytotoxicity (as shown in FIG. 6A), and ELISA (as shown in FIG. 6B) was used to detect the level of IFN-γ in cell supernatants.

Results: As shown in FIG. 6A: 1) Virus VG2003 or VG22001 alone can induce A549 cell death; 2) CD19-CAR-T cells or Mock-T cells alone can also induce A549 cell death; 3) VG22001 with CD19-αTfR and CD19-CAR-T cells together induced the highest level of cytotoxicity (while the virus VG2003 without CD19-αTfR and CD19-CAR-T cells together induced less cytotoxicity); and 4) The virus VG22001 or VG2003 and Mock T cells together induced less cytotoxicity than virus administered alongside CD19-CAR-T cells.

As shown in FIG. 6B: 1) Virus VG2003 or VG22001 alone can induce <10 pg/ml IFN-γ; 2) Mock-T cells alone or Mock T with VG2003 together can induce <10 pg/ml IFN-γ; 3) VG22001 or VG2003 and Mock T together can induce similar levels of IFN-γ (around 25 pg/ml); 4. VG22001 with CD19-αTfR and CD19-CAR-T cells together induce the highest level of IFN-γ (around 40 pg/ml)

Conclusions: Cytotoxicity and IFN-γ expression in A549 cells was most pronounced when the virus VG2201 (expressing CD19-αTfR) was co-cultured with CD19-CAR-T cells.

### Example 7

### Retargeting αHER2-ADC to Cells with Reduced Expression of HER2 on the Cell Surface Using a HER2-αTfR Adapter Molecule

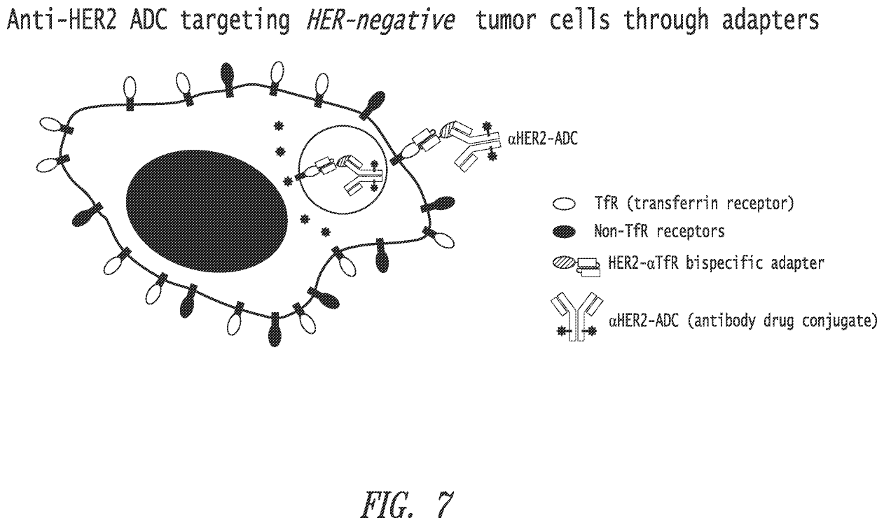

Objective: Antibody drug conjugates (ADCs) comprise an antibody against an antigen of interest, such as a tumor antigen, that is linked to a cytotoxic payload designed to destroy the antigen-bearing cell after the ADC is internalized. However, the tumor antigen landscape in clinical practice is heterogenous, with antigenic drift further altering the antigenic milieu within the tumor microenvironment subject to selection pressure from antitumor agents targeted to a specific antigen profile. The present invention allows for retargeting of antigen-specific ADCs (delivered either locally or systemically) to other cells within the tumor microenvironment that do not necessarily display the antigen recognized by said antigen-specific ADCs. Retargeting is accomplished by using an adapter molecule comprising a fragment of the antigen recognized by the ADC and an antibody targeting a ubiquitous broadly tumor-associated antigen such as the human transferrin receptor 1 (TfR1) (FIG. 7). Off-target effects are minimized by delivering the adapter intratumorally either by direct injection of the purified adapter protein, by injecting RNA encoding the adapter (as LNP-encapsulated mRNA, self-amplifying RNA, etc.), or by delivering the adapter as a secretable payload expressed by an oncolytic virus such as an oncolytic herpesvirus.

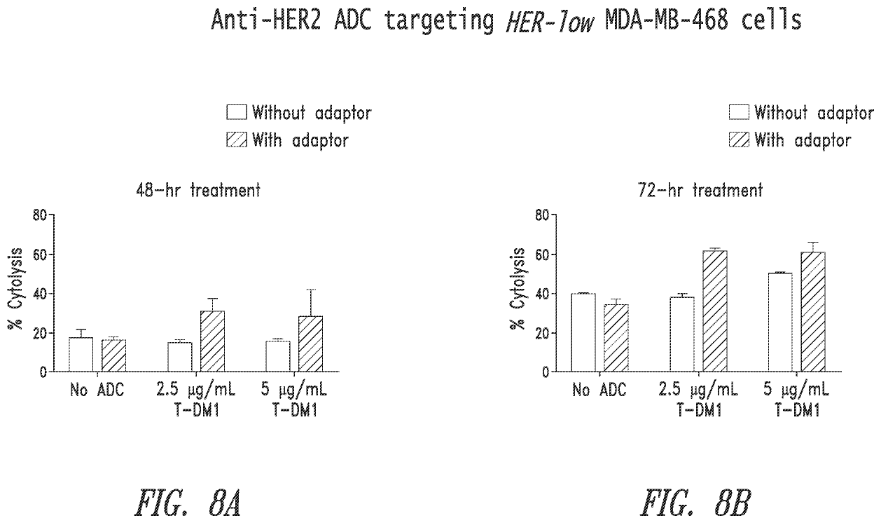

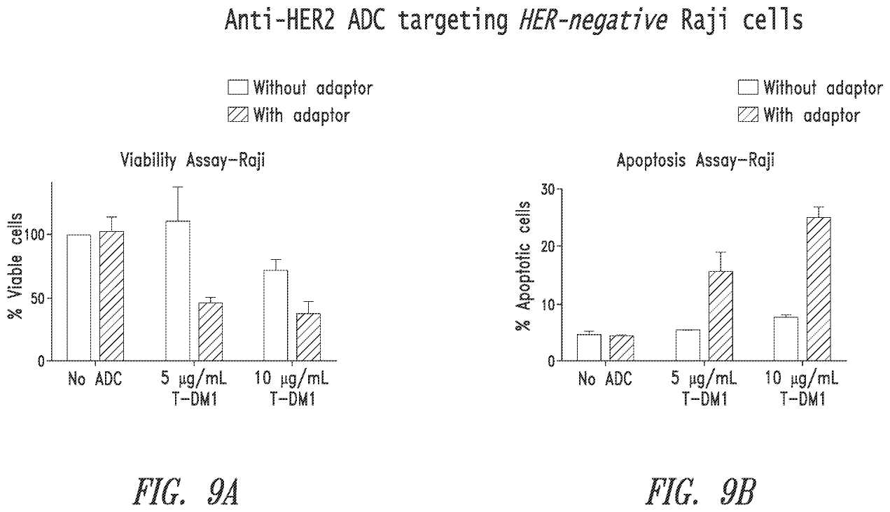

Study design: The adapter molecule HER2-αTfR was designed with one arm comprising the F12 scFv designed to target TfR1, and the other arm comprising the HER2 ectodomain. The FDA-approved anti-HER2 ADC ado-trastuzumab emtansine (T-DM1) comprised the humanized anti-HER2 antibody trastuzumab linked to the microtubule disrupting agent DM1. For testing T-DM1 retargeting to cells with low levels of HER2 expression, we seeded 2×10\hat{ }4 of HER2-low-expressing MDA-MB-468 cells in 96-well plates on Day 1, followed on Day 2 by incubation with 25 nM of the HER2-αTfR adapter at room temperature for 45 minutes. Anti-HER2 ADC T-DM1 was then added and incubated at 37° C. for either 48 hours or 72 hours. LDH assay was used to determine cellular cytotoxicity at the 48-hour (FIG. 8A) or the 72-hour (FIG. 8B) timepoints. For testing T-DM1 retargeting to HER2-negative cells, we seeded 2×10\hat{ }4 of HER2-negative Raji cells in 96-well plates on Day 1, followed on Day 2 by incubation with 25 nM of the HER2-αTfR adapter at room temperature for 45 minutes. Anti-HER2 ADC T-DM1 was then added and incubated at 37° C. for 48 hours. The Trypan blue exclusion method was used to determine cell viability (FIG. 9A), and flow cytometry was used to evaluate cell apoptosis via 7-AAD (FIG. 9B).

Results: Exposure to the adapter molecule HER2-αTfR approximately doubled the cell killing ability of the anti-HER2 ADC T-DM1 on both HER2-low-expressing and HER2-negative tumor cells at the 48-hour timepoint. Enhanced cytolysis, reduced cell viability, and increased apoptosis were observed in adapter-treated cells exposed to all tested dosages of T-DM1. Improved cytolysis at the 72-hour timepoint after treatment with the adapter molecule was also evident but less pronounced due to naturally higher baseline levels of cytolysis at that point, as demonstrated by the control samples which were not exposed to either T-DM1 or to the adapter.

Conclusions: The adapter molecule HER2-αTfR can successfully bind to the TfR1 receptor on both HER2-low-expressing MDA-MB-468 cells and HER2-negative Raji cells and retarget the HER2-specific ADC T-DM1 to potentiate ADC-mediated cell killing of adapter-decorated cells.

The following are additional exemplary embodiments of the present disclosure:
1. An oncolytic virus that directs expression of a soluble multivalent adapter protein (also referred to as a “multivalent engager protein”), wherein the multivalent adapter protein comprises a first domain comprising a fragment of an antigen, and, a second domain comprising a second antigen binding domain, wherein the second antigen binding domain specifically binds to a second tumor-associated antigen (e.g., a tumor-surface protein). Within various embodiments the invention an “antigen” refers to a protein or peptide sequence that can invoke an immune response. Representative examples include tumor associated antigens, protein or peptide sequences that can be bound by antibodies, protein or peptide sequences that can be bound by genetically engineered immune reactive cells, and non-human protein or peptide sequences. Within various embodiments the multivalent proteins disclosed herein can be, for example, bivalent or bispecific, trivalent or trispecific, quadrivalent or quadraspecific, and/or pentavalent or pentaspecific.
2. The oncolytic virus of embodiment 1, wherein the antigen is a first tumor-associated antigen. Within a preferred embodiment of the invention the second tumor-associated antigen that is targeted is more broadly expressed than the first tumor-associated antigen.
3. The oncolytic virus of embodiment 2, wherein the first tumor-associated antigen comprises a transmembrane protein.
4. The oncolytic virus of embodiment 2, wherein the first tumor-associated antigen is selected from the group consisting of CD19, BCMA, HER2, EGFR, IL13Ra2, CEA, EGFRvIII, Mesothelin, Claudin 18.2, PSCA, CAIX, AXL, PSMA, Folate receptor-alpha, MUC16, MUC1, ROR1, and OR2H1. Within other embodiments the tumor antigen is selected from the group consisting of AIM-2, AIM-3, ART1, ART4, B7-H3, B7-H6, BAGE, β1,6-N, β-catenin, B-cyclin, BMI1, BRAF, BRAP, C13orf24, C6orf153, C9orf112, CA-125, CABYR, CASP-8, cathepsin B, Cav-1, CD70, CD74, CDK-1, CEA, CEAmidkin, COX-2, CRISP3, CSAG2, CSPG4, CTAG2, CYNL2, DHFR, E-cadherin, EGFRvIII, EpCAM, EphA2/Eck, ESO-1, EZH2, FAP, FRα, Fra-1/Fosl 1, FTHL17, GAGE1, Ganglioside/GD2, GD3, GLEA2, GliI, GLUT1, GnT-V, GOLGA, gp75, gplOO, HER-2, HLA-A1+MAGE1, HSPH1, IL-11R, IL13Ralpha, IL13Ralpha2, ING4, Ki67, KIAA0376, Ku70/80, LDHC, Lewis-Y, UCAM, Livin, MAGE-A1, MAGE-2, MAGE-A3, MAGE-B6, MAPPK1, MART-1, Mesothelin, MICA, MRP-3, MUC-1, Muc16, MUM-1, Nestin, NKG2D, NKTR, NLRP4, NSEP1, NY-ES-01, OLIG2, p53, PAP, PBK, PRAME, PROX1, PSA, PSCA, PSMA, ras, RBPSUH, ROR1, RTN4, SART1, SART2, SART3, SOX10, SOX11, SOX2, SPANXA1, SSX2, SSX4, SSX5, Survivin, TAG72, TfR1, TNKS2, TPR, TRP-1, TRP-2, TSGA10, TSSK6, TULP2, Tyrosinase, U2AF1L, UPAR, VEGFR2, WT-1, XAGE2, and ZNF165. Within yet further embodiments the tumor antigen is selected from the group consisting of an antigen provided in FIGS. 4A to 4WW.
5. The oncolytic virus of embodiment 2, wherein the first tumor-associated antigen comprises an ectodomain of the first tumor-associated antigen.
6. The oncolytic virus of embodiment 1, wherein the antigen is a protein or protein fragment of non-human origin.
7. The oncolytic virus of embodiment 6, wherein said protein or protein fragment is of non-human vertebrate origin; invertebrate origin; fungal origin; arthropod origin; mollusk origin; cnidarian origin; poriferan origin; archaean origin; bacterial origin; protist origin; plant origin (e. g., gluten); or, of viral origin (e. g., a viral surface protein or a fragment thereof).
8. The oncolytic virus of embodiment 1, wherein the second tumor-associated antigen is a cell surface antigen that is more broadly expressed in solid tumors relative to the first tumor-associated antigen.
9. The oncolytic virus of embodiment 8, wherein the second tumor-associated antigen is transferrin receptor 1 (TfR1) or glucose transporter 1 (GLUT1).
10. The oncolytic virus of embodiment 8, wherein the second tumor-associated antigen is TfR1 and the second binding domain is an antibody or fragment. Within preferred embodiments the second binding domain is a fragment of the H7 or F12 monoclonal antibody.
11. The oncolytic virus of embodiment 1, wherein the second antigen binding domain comprises an antibody.
12. The oncolytic virus of embodiment 11, wherein the antibody is an scFv fragment.
13. The oncolytic virus of embodiment 1, wherein the second antigen binding domain specifically binds the second tumor-associated antigen in a pH-dependent manner.
14. The oncolytic virus of embodiment 1, wherein the first domain and the second domain are joined by a linker.
15. The oncolytic virus of embodiment 14, wherein the linker comprises one or more repeats of the amino acid sequence GGGGS.
16. The oncolytic virus of embodiment 1, wherein the virus is a recombinant oncolytic virus selected from the group consisting of oncolytic herpes virus 1 (HSV-1), oncolytic herpes virus 2 (HSV-2), oncolytic adenovirus, and oncolytic vaccinia virus.
17. The oncolytic virus of embodiment 1, wherein the virus further directs expression of one or more immunomodulators selected from the group consisting of IL-12, IL-15, IL-15RA1 and IL-18.
18. The oncolytic virus of embodiment 1, wherein the virus is modified to enable tumor-specific viral replication and/or tumor-specific expression of the multivalent adapter protein.
19. A multivalent adapter protein, wherein the multivalent adapter protein comprises a first domain comprising a fragment of an antigen, and, a second domain comprising a second antigen binding domain, wherein the second antigen binding domain specifically binds to a second tumor-associated antigen (e.g., a tumor-surface protein). Within various embodiments the invention an “antigen” refers to a protein or peptide sequence that can invoke an immune response. Representative examples include tumor associated antigens, protein or peptide sequences that can be bound by antibodies, protein or peptide sequences that can be bound by genetically engineered immune reactive cells, and non-human protein or peptide sequences. Within various embodiments the multivalent proteins disclosed herein can be, for example, bivalent or bispecific, trivalent or trispecific, quadravalent or quadraspecific, and/or pentavalent or pentaspecific.
20. The multivalent adapter protein of embodiment 19, wherein the antigen is a first tumor associated antigen.
21. The multivalent adapter protein of embodiment 20, wherein the first tumor-associated antigen comprises a transmembrane protein.
22. The multivalent adapter protein of embodiment 21, wherein the first tumor-associated antigen is selected from the group consisting of CD19, BCMA, HER2, EGFR, IL13Ra2, CEA, EGFRvIII, Mesothelin, Claudin 18.2, PSCA, CAIX, AXL, PSMA, Folate receptor-alpha, MUC16, MUC1, ROR1, and OR2H1. Within other embodiments the tumor antigen is selected from the group consisting of AIM-2, AIM-3, ART1, ART4, B7-H3, B7-H6, BAGE, β1,6-N, β-catenin, B-cyclin, BMI1, BRAF, BRAP, C13orf24, C6orf153, C9orf112, CA-125, CABYR, CASP-8, cathepsin B, Cav-1, CD70, CD74, CDK-1, CEA, CEAmidkin, COX-2, CRISP3, CSAG2, CSPG4, CTAG2, CYNL2, DHFR, E-cadherin, EGFRvIII, EpCAM, EphA2/Eck, ESO-1, EZH2, FAP, FRα, Fra-1/Fosl 1, FTHL17, GAGE1, Ganglioside/GD2, GD3, GLEA2, GliI, GLUT1, GnT-V, GOLGA, gp75, gplOO, HER-2, HLA-A1+MAGE1, HSPH1, IL-11Ra, IL13Ralpha, IL13Ralpha2, ING4, Ki67, KIAA0376, Ku70/80, LDHC, Lewis-Y, LICAM, Livin, MAGE-A1, MAGE-2, MAGE-A3, MAGE-B6, MAPPK1, MART-1, Mesothelin, MICA, MRP-3, MUC-1, Muc16, MUM-1, Nestin, NKG2D, NKTR, NLRP4, NSEP1, NY-ES-01, OLIG2, p53, PAP, PBK, PRAME, PROX1, PSA, PSCA, PSMA, ras, RBPSUH, ROR1, RTN4, SART1, SART2, SART3, SOX10, SOX11, SOX2, SPANXA1, SSX2, SSX4, SSX5, Survivin, TAG72, TfR1, TNKS2, TPR, TRP-1, TRP-2, TSGA10, TSSK6, TULP2, Tyrosinase, U2AF1L, UPAR, VEGFR2, WT-1, XAGE2, and ZNF165. Within yet further embodiments the tumor antigen is selected from the group consisting of an antigen provided in FIGS. 4A to 4WW.
23. The multivalent adapter protein of embodiment 20, wherein the fragment of the first tumor-associated antigen comprises an ectodomain of the first tumor-associated antigen.
24. The multivalent adapter protein of embodiment 19, wherein the antigen is a protein or protein fragment of non-human origin.
25. The multivalent adapter protein of embodiment 24, wherein said protein or protein fragment is of non-human vertebrate origin; invertebrate origin; fungal origin; arthropod origin; mollusk origin; cnidarian origin; poriferan origin; archaean origin; bacterial origin; protist origin; plant origin (e. g., gluten); or, of viral origin (e. g., a viral surface protein or a fragment thereof).
26. The multivalent adapter protein of embodiment 19, wherein the second tumor-associated antigen is a cell surface antigen that is more broadly expressed in solid tumors relative to the first tumor-associated antigen.
27. The multivalent adapter protein of embodiment 26, wherein the second tumor-associated antigen is TfR1 and the second binding domain is an antibody or fragment. Within preferred embodiments the second binding domain is a fragment of the H7 or F12 monoclonal antibody.
28. The multivalent adapter protein of embodiment 19, wherein the second antigen binding domain comprises an antibody.
29. The multivalent adapter protein of embodiment 28, wherein the antibody is an scFv fragment.
30. The multivalent adapter protein of embodiment 19, wherein the second antigen binding domain specifically binds the second tumor-associated antigen in a pH-dependent manner.
31. The multivalent adapter protein of embodiment 19, wherein the first domain and the second domain are joined by a linker.
32. The multivalent adapter protein of embodiment 31, wherein the linker comprises one or more repeats of the amino acid sequence GGGGS.
33. The multivalent adapter protein of embodiment 19, wherein said multivalent adapter protein is soluble.
34. An ADC, chimeric antigen receptor modified immune cell, or T cell receptor modified immune cell, wherein said ADC or immune cell is modified to recognize a protein or protein fragment of non-human origin, or a tumor associated antigen. Within further embodiments the ADC, chimeric antigen receptor modified immune cell, or T cell receptor modified immune cell comprises a second antigen binding domain, wherein the second antigen binding domain specifically binds a fragment of a first tumor-associated antigen.
35. The chimeric antigen receptor modified immune cell, or T cell receptor modified immune cell of claim 34, wherein said cell is modified from a T cell, a NK cell, a macrophage cell, a neutrophil cell, or a B cell.
36. The ADC, chimeric antigen receptor modified immune cell, or T cell receptor modified immune cell of claim 34 wherein said protein or protein fragment is of non-human vertebrate origin; invertebrate origin; fungal origin; arthropod origin; mollusk origin; cnidarian origin; poriferan origin; archaean origin; bacterial origin; protist origin; plant origin (e. g., gluten); or, of viral origin (e. g., a viral surface protein or a fragment thereof).
37. A method of inhibiting growth of a tumor in a subject comprising administering to the subject a therapeutically effective amount of a first immunotherapeutic agent comprising the oncolytic virus of any one of embodiments 1-18 and a second immunotherapeutic agent comprising an ADC, chimeric antigen receptor modified immune cell, or T cell receptor modified immune cell according to any one of embodiments 34 to 36. Within various embodiments the cell which is genetically modified to express a chimeric antigen receptor or modified T cell receptor is a T-cell, a NK cell, a macrophage, a neutrophil, a B-cell, or a TCR-T cell.
38. A method of inhibiting growth of a tumor in a subject comprising administering to the subject a therapeutically effective amount of a first immunotherapeutic agent comprising the oncolytic virus of any one of embodiments 1-18, and a second immunotherapeutic agent comprising an antibody drug conjugate (ADC).
39. A method of inhibiting growth of a tumor in a subject comprising administering to the subject a therapeutically effective amount of a first immunotherapeutic agent comprising the multivalent engager of any one of embodiments 19 to 33 and a second immunotherapeutic agent comprising an ADC, or chimeric antigen receptor modified immune cell, or T cell receptor modified immune cell according to any one of embodiments 34 to 36. Within various embodiments the cell which is genetically modified to express a chimeric antigen receptor or modified T cell receptor is a T-cell, a NK cell, a macrophage, a neutrophil, a B-cell, or a TCR-T cell.
40. A method of inhibiting growth of a tumor in a subject comprising administering to the subject a therapeutically effective amount of a first immunotherapeutic agent comprising the multivalent engager of any one of embodiments 19 to 33, and a second immunotherapeutic agent comprising an antibody drug conjugate (ADC). Within a preferred embodiment of the invention the multivalent adapter protein is delivered directly without a vector. Within another preferred embodiment of the invention the multivalent adapter protein is delivered through a vector. Within one embodiment the multivalent adapter protein is encoded by a nucleic acid (e.g., mRNA) and delivered, e.g., via self-amplifying RNA, and/or a lipid nanoparticle. Within various embodiments the vector is an oncolytic virus according to any one of embodiments 1 to 18.
41. The method of any one of embodiments 37 to 40, wherein the first immunotherapeutic agent is administered to the subject prior to administration of the second immunotherapeutic agent.
42. The method of any one of embodiments 37 to 40, wherein the subject is a human.
43. The method of any one of embodiments 37 to 40, wherein the tumor is a solid tumor and/or a diffuse tumor.
44. The method of embodiment 43 wherein the solid tumor is selected from the group consisting of lung, stomach, prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, melanoma and urogenital tract.
45. A method of treating a patient having a cancer, the method comprising administering to the patient a therapeutically effective amount of a first pharmaceutical composition comprising the oncolytic virus of any one of embodiments 1-18 and a second pharmaceutical composition comprising an ADC, or chimeric antigen receptor modified immune cell, or T cell receptor modified immune cell according to any one of embodiments 34 to 36.
46. A method of treating a patient having a cancer, the method comprising administering to the patient a therapeutically effective amount of a first pharmaceutical composition comprising the oncolytic virus of any one of embodiments 1-18, and a second pharmaceutical composition comprising an antibody drug conjugate (ADC).
47. A method of treating a patient having a cancer, the method comprising administering to the patient a therapeutically effective amount of a first pharmaceutical composition comprising the multivalent adapter protein according to any one of embodiments 19 to 33, and a second pharmaceutical composition comprising an ADC, or chimeric antigen receptor modified immune cell, or T cell receptor modified immune cell according to any one of embodiments 34 to 36.
48. A method of treating a patient having a cancer, the method comprising administering to the patient a therapeutically effective amount of a first pharmaceutical composition comprising the multivalent adapter protein of embodiments 19 to 33, and a second pharmaceutical composition comprising an antibody drug conjugate (ADC). Within a preferred embodiment of the invention the multivalent adapter protein is delivered directly without a vector. Within another preferred embodiment of the invention the multivalent adapter protein is delivered through a vector. Within one embodiment the multivalent adapter protein is encoded by a nucleic acid (e.g., mRNA) and delivered, via self-amplifying RNA, and/or a lipid nanoparticle. Within a further preferred embodiment, the vector is a virus such as an oncolytic virus according to any one of embodiments 1 to 18.
49. The method of embodiment 48, wherein the first pharmaceutical composition is administered to the subject prior to administration of the second pharmaceutical composition.
50. The method of embodiment 48, wherein the subject is a human.
51. The method of embodiment 48, wherein the cancer is a solid tumor and/or a diffuse tumor.
52. The method of embodiment 48 wherein said cancer is a cancer selected from the group consisting of cancers of the lung, stomach, prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, melanoma and urogenital tract.
53. The method of embodiment 48, wherein the step of administering comprises intratumoral injection. Within further embodiments, only the adapter is administered intratumorally.
54. A kit, comprising at least two of: a) an oncolytic virus according to any one of embodiments 1-18; b) a multivalent adapter protein according to any one of embodiments 19-33; and c) an ADC, chimeric antigen receptor modified immune cell, or T cell receptor modified immune cell according to any one of embodiments 34 to 36.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising,” “including,” “containing,” etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. An oncolytic virus that directs expression of a soluble multivalent adapter protein, wherein the multivalent adapter protein comprises a first domain comprising a fragment of a first tumor-associated antigen, and, a second domain comprising a second antigen binding domain, wherein the second antigen binding domain specifically binds to a second tumor-associated antigen.

2. (canceled)

3. The oncolytic virus of claim 1, wherein the first tumor-associated antigen is selected from the group consisting of CD19, BCMA, HER2, EGFR, IL13Ra2, CEA, EGFRvIII, Mesothelin, Claudin 18.2, PSCA, CAIX, AXL, PSMA, Folate receptor-alpha, MUC16, MUC1, ROR1, and OR2H1.

4. The oncolytic virus of claim 1, wherein the fragment of the first tumor-associated antigen comprises an ectodomain of the first tumor-associated antigen.

5. The oncolytic virus of claim 1, wherein the second tumor-associated antigen is a cell surface antigen that is more broadly expressed in solid tumors relative to the first tumor-associated antigen.

6. The oncolytic virus of claim 5, wherein the second tumor-associated antigen is transferrin receptor 1 (TfR1) or glucose transporter 1 (GLUT1).

7. The oncolytic virus of claim 5, wherein the second tumor-associated antigen is TfR1 and the antibody or fragment thereof is the H7 or F12 monoclonal antibody.

8. The oncolytic virus of claim 1, wherein the second antigen binding domain comprises an antibody, wherein the antibody is an scFv fragment.

9. (canceled)

10. The oncolytic virus of claim 1, wherein the second antigen binding domain specifically binds the second tumor-associated antigen in a pH-dependent manner.

11. The oncolytic virus of claim 1, wherein the first domain and the second domain are joined by a linker, wherein the linker comprises one or more repeats of the amino acid sequence GGGGS.

12. (canceled)

13. The oncolytic virus of claim 1, wherein the virus is a recombinant oncolytic virus selected from the group consisting of oncolytic herpes virus 1 (HSV-1), oncolytic adenovirus, and oncolytic vaccinia virus.

14. The oncolytic virus of claim 1, wherein the virus further directs expression of one or more immunomodulators selected from the group consisting of IL-12, IL-15, IL-15RA1 and IL-18.

15. The oncolytic virus of claim 1, wherein the virus is modified to enable tumor-specific viral replication and/or tumor-specific expression of the multivalent adapter protein.

16. A method of inhibiting growth of a tumor in a subject comprising administering to the subject a therapeutically effective amount of a first immunotherapeutic agent comprising the oncolytic virus of claim 1 and a second immunotherapeutic agent comprising an ADC, or, a T cell genetically modified to express a chimeric antigen receptor (CAR), wherein the CAR comprises a second antigen binding domain, a transmembrane domain, and an intracellular signaling domain, wherein the second antigen binding domain specifically binds the fragment of the first tumor-associated antigen of the oncolytic virus.

17. The method of claim 16, wherein the first immunotherapeutic agent is administered to the subject prior to administration of the second immunotherapeutic agent.

18. The method of claim 16, wherein the subject is a human.

19. The method of claim 16, wherein the tumor is a solid tumor.

20. The method of claim 19 wherein the solid tumor is selected from the group consisting of lung, stomach, prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, melanoma and urogenital tract.

21. A method of treating a patient having a cancer, the method comprising administering to the patient a therapeutically effective amount of a first pharmaceutical composition comprising the oncolytic virus of claim 1 and a second pharmaceutical composition comprising an ADC, or, a T cell genetically modified to express a chimeric antigen receptor (CAR), wherein the CAR comprises a second antigen binding domain, a transmembrane domain, and an intracellular signaling domain, wherein the second antigen binding domain specifically binds the fragment of the first tumor-associated antigen of the oncolytic virus.

22. The method of claim 21, wherein the first pharmaceutical composition is administered to the subject prior to administration of the second pharmaceutical composition.

23. The method of claim 21, wherein the subject is a human.

24. The method of claim 21, wherein the cancer is a solid tumor and/or a diffuse tumor.

25. The method of claim 21 wherein said cancer is a cancer selected from the group consisting of cancers of the lung, stomach, prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, melanoma and urogenital tract.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)


SEQUENCE LISTING (8 sequences)

SEQ ID NO: 1 - protein, 547 aa, Homo sapiens
MEFGLSWVFLVAILKGVQPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKGGGGSGGGGSGGGGSQVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSSGGGGSGGGGSGGGGSSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVL

SEQ ID NO: 2 - protein, 18 aa, Homo sapiens
MEFGLSWVFLVAILKGVQ

SEQ ID NO: 3 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 4 - protein, 15 aa, Homo sapiens
GGGGSGGGGSGGGGS

SEQ ID NO: 5 - protein, 242 aa, Homo sapiens
QVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSSGGGGSGGGGSGGGGSSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVL

SEQ ID NO: 6 - protein, 591 aa, synthetic construct
  REGION 1..591: note = HSV-human fusion protein
MEFGLSWVFLVAILKGVQCGGLINKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYGAPGGGGSGGGGSGGGGSQVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSSGGGGSGGGGSGGGGSSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG

SEQ ID NO: 7 - protein, 306 aa, Herpes simplex virus
KYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPY

SEQ ID NO: 8 - protein, 241 aa, Homo sapiens
QLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSSGGGGSGGGGSGGGGSSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG